(19) United States
(12) Patent Application Publication
(10) Pub. No.: US 2019/0174768 A1
(43) Pub. Date: Jun. 13, 2019

(54) USE OF A CELLULAR EXTRACT OF ONE OR MORE MICROALGAE OF THE AMPHIDINIUM GENUS, FOR ITS FUNGICIDAL AND/OR BACTERICIDAL ACTIVITY ON FUNGI, OOMYCETES AND/OR PATHOGENIC BACTERIA OF PLANTS AND CULTURE SEEDS

(71) Applicant: Immunrise Biocontrol France, Pessac (FR)

(72) Inventors: Odon Thiebeauld de la Crouee, Bourg la Reine (FR); Yann Thomas, Ermont (FR)

(73) Assignee: Immunrise Biocontrol France, Pessac (FR)

(21) Appl. No.: 16/308,111

(22) PCT Filed: Jun. 8, 2017
(86) PCT No.: PCT/EP2017/064046

§ 371 Date: Dec. 7, 2018

(30) Foreign Application Priority Data
Jun. 8, 2016 (FR) 1655263

(51) Int. Cl. A01N 65/03; C12N 1/12

(52) U.S. Cl. CPC A01N 65/03; C12N 1/12

(57) ABSTRACT

The invention relates to a cellular extract of one or more microalgae of the Amphidinium genus and to the uses thereof for its fungicidal and/or bactericidal activity on fungi, oomycetes and/or pathogenic bacteria of plants and culture seeds.

The invention relates to the field of antifungal and antibactericidal agents for seeds.

In order to cope with world population growth, the period following the Second World War saw the advent of the “Green Revolution”, a set of modern programmes concerning varietal selection, irrigation, fertilizers and synthetic pesticides, the purpose of which was to control soil fertility and pathogenic organisms. These various elements have thus made it possible to almost triple global food production in the space of some forty years. Today, the agricultural challenge is to feed 9 billion people by 2050 and to continue to increase production per unit area in the context of increasingly limited resources and increasing constraints. For example, potential losses due to biotic stresses and the absence of crop protection methods could amount to more than half of cereal production. To limit losses, conventional plant protection products are thus an integral part of crop protection worldwide. However, these chemicals have a strong negative impact on human health and the environment, leading to the use of other means of controlling infectious diseases, such as biocontrol (all the plant protection methods using natural mechanisms). The object of this patent is to exploit unicellular algae, derived from phytoplankton, as a source of new natural molecules capable of acting as an “organic pesticide” by directly affecting the survival of phytopathogens infecting crops of major agronomic importance, such as wheat and grapevine.

### Fusarium Diseases

In Europe, several diseases affecting wheat (Triticum aestivum) are responsible for yield losses and lower health quality of grains. One of the most important is Septoria leaf blotch (Septoria spp.). Fusarium wilt is associated with a species complex consisting of two genera of phytopathogenic fungi, Fusarium and Microdochium (1). These two genera include about 19 species capable of inducing Fusarium head blight in wheat. The most common species in Europe are F. graminearum, F. culmorum, F. avenaceum, F. poae, M. nivale and M. majus. The genus Fusarium belongs to the division Ascomycota and the family Nectriaceae. The genus Microdochium belongs to the family Tuberculariaceae and includes two species, M. nivale and M. majus, causing the same symptoms on the ear and leaves as Fusarium. Several Fusarium species, of which Fusarium graminearum is the most represented, can be found together in a given region or plot or ear, thus forming the Fusarium complex. The severity, incidence and prevalence of each species vary by geographical location, climatic variations and cultural practices. The presence of several of these species on the same ear is likely to modify their equilibrium and their toxin production dynamics.

Fusarium head blight of wheat can devastate a crop a few weeks before harvest. It may be associated with high yield losses (grain abortion and low weight), a reduction in germinative quality or a decrease in quality due to the presence of toxins in the grains. Indeed, fungi of the genus Fusarium, but not of the genus Microdochium, are capable of producing toxic secondary metabolites, mycotoxins, whose presence increases the incidence of the disease on agricultural production and constitutes a major economic and public health problem. The main means of controlling Fusarium wilt include cultivation practices, varietal resistance and chemical control. At present, few wheat varieties are resistant to Fusarium wilt. However, there are tolerant varieties with partial resistance levels that limit yield losses and toxin accumulation in harvests. Once the crop is established, chemical control is possible but of limited efficacy. The diversity of pathogens and their different sensitivity to active substances complicate this control. For example, fungi of the genus Fusarium are sensitive to triazoles while fungi of the genus Microdochium are sensitive to strobilurins.

### Septoria Diseases

Septoria blotch is a wheat disease which is responsible for significant yield losses and causes the most economic losses worldwide, notably in humid temperate regions. Two main forms of Septoria blotch can be distinguished: glume blotch (Phaeosphaeria nodorum) and leaf blotch (Mycosphaerella graminicola). In France, glume blotch is mainly present in continental areas, while leaf blotch is mainly present in the northwest and coastal regions where the fungus finds climatic conditions favourable to its development. Symptoms caused by M. graminicola appear successively in the form of chlorosis, light green spots, before evolving into brownish spots called necrotic lesions. These necrotic lesions eventually merge (coalescence). Next, pycnidia, black fruiting bodies barely visible to the naked eye, appear on these necrotic lesions. The harmfulness of Septoria blotch in terms of loss of photosynthesis, growth or yield has been studied by several research teams. A qualitative harmfulness expressing the impact of the disease on the protein content of harvested grains can thus be established.

M. graminicola is a hemibiotrophic fungus that establishes a first biotrophic phase where infection occurs on living tissues followed by the necrotrophic phase during which the fungus expresses toxins producing death of colonized tissues. Depending on environmental conditions, M. graminicola reproduction is either sexual (ascospore production) or asexual (pycnidiospore production). Ascospores, spread by wind over long distances, contribute to the survival of the fungus in the absence of a host plant and are considered the main source of primary inoculum to initiate the disease. Pycnidiospores, in turn, are mostly produced during the epidemic phase of the disease over several successive infectious cycles. These spores are splashdispersed over short distances by the action of raindrops. The decrease in potential yields is all the greater as the last leaves under the ear involved in grain filling are severely affected by the disease. Yield losses due to Septoria blotch have been estimated at 1-2 tha⁻¹ on average, with cases of up to 3-3.5 tha⁻¹, representing a 40% decrease in yields.

Methods for controlling M. graminicola are based on the use of fungicides and resistant cultivars. However, in recent years there has been a significant loss in fungicide efficacy due to a strong selection of pathogens with, for example, resistance to the family of strobilurins as well as a recent loss in the field efficacy of triazoles.

### Grapevine Diseases

Today, the grapevine is grown worldwide and plays a central role in the economies of many countries. It is consumed as table grapes and juice, but its main exploitation rests on the wine industry. The European Union is the world's largest wine producer and the world's largest exporter of wine products. The sector thus contributes around 15 billion euros per year to the European Union economy (www.ceev.be). In 2010, French vineyards covered nearly 865 000 hectares, or nearly 3% of arable land, and made France the world's leading wine producer with 51.1 million hectolitres. Grapevines must cope with many pathogenic attacks, including cryptogamic diseases. When they affect the lignified parts of the plant they are referred to as “wood disease”, notable examples being esca, black dead arm and eutypiosis. Fungi that infect berries and herbaceous parts of the grapevine (leaves, stems, etc.) induce “cryptogamic diseases of the foliage” including grey rot, black rot, downy mildew and powdery mildew.

### Esca

While downy mildew, powdery mildew and grey rot are the three main cryptogamic diseases affecting vineyards worldwide, wood diseases caused by fungal agents are becoming limiting factors in grape production. Winegrowers are currently confronted with two major problems concerning these wood diseases: the lack of control methods and a profound lack of knowledge of the various biotic and abiotic factors.

The most common fungal species worldwide for esca disease are the ascomycetes Diplodia seriata, Diplodia mutila, Neofusicoccum parvum and Neofusicoccum luteum. In France, the most isolated species are Diplodia seriata and Botryosphaeria dothidea. Many other fungi, including some pathogens, are frequently isolated from wood necrosis in plants with esca. This is the case of Eutypa lata, the agent responsible for eutypiosis. This disease has two forms: the slow form and the apoplectic form. Foliar symptoms are characteristic of the slow form even though they may be present in the apoplectic form. The slow form is characterized by specific foliar colourings: yellowish interveinal spots on white grape varieties and red-bordered interveinal spots on black grape varieties, the veins remaining green. These spots gradually evolve into browning and drying. Foliar symptoms of the slow form can be visible one year on a grapevine stock and disappear the next year. The apoplectic form is characterized by a rapid drying of the aerial organs, twigs, leaves and bunches of part or all of the grapevine stock. This symptom usually occurs when summers are hot, resulting in the death of the grapevines in only a few days without warning symptoms. The variety of inoculum sources and the very slow and invisible development of fungi in grapevine wood make it very difficult to implement control methods. In addition, changes in the regulation of plant protection products at the European level have led to the banning of sodium arsenite based chemicals because of the carcinogenic effects on humans and the high toxicity of these products to the environment. A great deal of research is being carried out throughout the world to test new molecules that can be used in nurseries or vineyards.

### Grey Rot

Grey rot is a cryptogamic disease caused by an ascomycete fungus called Botrytis cinerea. It belongs to the class Leotiomycetes, the order Helotiales and the family Sclerotiniaceae. B. cinerea is a necrotrophic fungus capable of colonizing healthy, already infected plant tissues as well as dead tissues (saprophytism). On the leaf, symptoms appear as brown spots with greyish felting on the underside (fruiting of the fungus) that tend to increase and invade the entire leaf blade. The bunches can be affected before flowering and dry out. They are especially sensitive at the veraison stage where there is development of a brown colouring of the berries of the white grape varieties and the appearance of a thick grey felt. Conidia are spread by wind and enter herbaceous organs directly or through wounds. This is why the bursting of the berries due to downy mildew favours infections by B. cinerea. This disease not only causes yield losses of up to 40% (Viniflhor, 2006 data) but also degrades the organoleptic qualities of wines. Nevertheless, Botrytis cinerea is also responsible for the “noble rot” necessary to obtain certain sweet wines.

### Downy Mildew

The two diseases that currently affect vineyards most severely are downy mildew and powdery mildew. The agent responsible for downy mildew, the oomycete Plasmospora viticola belonging to the order Peronosporales, is an obligate parasite; to stay alive and multiply, it is obligated to propagate on surviving grapevine leaves. P. viticola attacks all herbaceous tissues of the grapevine as well as the bunches. It causes defoliation, browning and drying of berries and stems. In the absence of treatment and under favourable climatic conditions, downy mildew can devastate up to 75% of the season's harvest.

The life cycle of P. viticola includes a sexual and an asexual phase. The asexual phase leads to the production of spores necessary for secondary infections and for dispersal of the pathogen over a short distance, while the sexual phase produces quiescent and cold-resistant oospores enabling overwintering and primary infections. The first macroscopic evidence of downy mildew in a vineyard is the appearance of pale yellow, irregular spots (oil spots) expanding on the upper or adaxial surface of the leaves. As the internal colonization of the mycelium progresses, the development of white cottony growths on the underside in correspondence with the oil spots becomes more pronounced. In advanced infections these symptoms are accompanied by dead brown tissue. Downy mildew is mainly controlled by preventive measures using fungicide sprays. While it is possible to stop an attack, the damage, once caused on inflorescences and clusters, is irreparable.

### Powdery Mildew

Grapevine powdery mildew (Erysiphe necator) is an obligate biotrophic ascomycete belonging to the order Erysiphales. The fungus colonizes the surface of all the green organs of the grapevine, especially the upper surface of the leaves, and spreads to the berries. A sexual phase characterized by the production of cleistothecia containing ascospores may alternate with an asexual phase leading to the formation of conidiophores bearing conidia. During the winter resting phase of the grapevine, the fungus survives as hyphae in the dormant buds or as cleistothecia on the surface of the plant. The spores contained in the cleistothecia will be released in the spring to germinate on the surface of buds and young leaves. A primary hypha develops on the leaf surface, then an increasingly complex and branched mycelial network covers the leaf surface. Subsequently, conidiophores differentiate from the mycelium at the beginning of the sporulation stage and colonize other green tissues of the plant leading to secondary infections.

The presence of mycelium and conidiophores carrying conidia on the surface of infected host tissues gives a greyish-white powdery appearance. White felting develops on the flower buds, which dry out. Only young berries with a sugar content of <8% are susceptible to powdery mildew. All leaf surfaces can be susceptible to infection, regardless of their age. Young infected leaves first turn dark green and then deform and become stunted. The upper surface of the leaves may have lighter, chlorotic spots similar to the oil spots of downy mildew. At present, the main means of controlling the diseases that affect vineyards most severely is the use of large amounts of pesticides and fungicides. Sanitary pressure is therefore particularly strong in viticulture.

Fungicidal treatments intended to control mainly downy mildew and powdery mildew are applied on a precise schedule to prevent damage due to the appearance of disease. The European Union (EU) uses about 68 000 tonnes of fungicides per year to control grapevine diseases, which represents 65% of the fungicides used in agriculture, while only 3.3% of the EU's useful agricultural area is occupied by grapevines (Eurostat, 2007). In order to limit the strong pressure of chemicals on the environment and health, it is necessary to isolate molecules of natural origin that will play a role in protecting crops against infectious diseases in order to eventually replace the chemical plant protection products used to date.

### Apple Scab

Scab, along with brown rot and powdery mildew, is one of the main fungal diseases of apple trees (genus Malus). It is caused by an ascomycete fungus called Venturia inaequelis, of which there are several thousand strains, causing black or brown lesions on the surface of leaves, buds or fruits and sometimes even on the wood. Fruits and the lower part of the leaves are particularly sensitive to it.

The fungus overwinters on the leaves that fall from infected trees, in the form of perithecia. In spring, when the buds open, the perithecia fill with ascospores. Ascospores are ejected into the air of the orchard on humid days and reach the trees through air movement. This ascospore discharge begins at bud burst and continues for 6 to 10 weeks, most often until the end of June. When the ascospores reach the foliage and the leaves are wet for a period of time, they germinate and penetrate the leaves, resulting in a primary infection. Depending on humidity and temperature conditions, the fungal infection becomes visible in one to three weeks on the different parts of the tree. Dark olive or brown spots of about 5 mm appear on the leaves and may sometimes cover the entire leaf. Infected flowers may fall off. Fruit infection is first identified by grey spots on the stem.

Following the primary infection and for the rest of the summer, the fungus develops and produces conidia, which are another form of reproductive structure. When the conidia escape, there is a secondary infection. The conidia can infect any part of the tree and those produced in late summer can even grow on stored fruit. Heavy rain disperses the conidia.

The disease rarely kills its host but can significantly reduce (up to 100%) fruit quality and production in the absence of fungicide treatment. After the preventive measures of collecting fallen leaves in the fall, the control strategy requires effective action in the spring to prevent released spores from infecting or developing on the trees. The traditional method of protection was to start fungicide application at bud burst and repeat applications every seven days or so until late June to protect new shoots. Apple orchards are the most heavily treated with fungicides and insecticides with an average of 28.8 fungicide treatments per year, 19 of which are dedicated to scab (INRA data).

### Microalgae

Molecules of natural origin having a new mechanism of action and capable of circumventing the resistance developed by pathogens have a major future for the development of new environmentally friendly plant protection products. The oceans represent a considerable variety of organisms (bacteria, microalgae, algae, vertebrate and invertebrate animals) which are a source of new bioactive molecules and which are still under-exploited (2). For example, marine microorganisms accumulate bioactive secondary metabolites whose unique structure is not found in terrestrial organisms. These metabolites therefore potentially represent new molecules of interest. Some substances derived from marine organisms have been described as having antifungal activity or natural defence substance activity, but the search for these molecules is still very underdeveloped (3).

Microalgae are unicellular organisms that play a key role in aquatic ecosystems. Producing organic material, they play an important ecological role as they represent the bottom of the marine environment food chain. However, their incredible ability to colonize all the world's oceans suggests that they have probably developed effective strategies to control pathogens, notably via the production of natural pesticides. For example, the abundant proliferation in coastal areas of microalgae producing biotoxins is responsible for the formation of harmful algal blooms (HABs) with a significant impact on the trophic cascade.

Among microalgae, dinoflagellates, belonging to the order Gymnodiniales and the family Gymnodiniacae, are present in temperate and tropical marine waters living in free form or in symbiosis with invertebrates (for example, corals). Dinoflagellates synthesize a large number of secondary metabolites called polyketides (compounds with biological or pharmacological activity that may be toxic to confer a survival advantage), several of which have been characterized, including those responsible for HABs (4). For example, the model species of dinoflagellates, Amphidinium carterae, produces a profusion of different bioactive compounds, many of which have the potential to be developed as therapeutic agents (5). The polyketides produced by Amphidinium species are extremely diverse in structure and form three categories: macrolides, linear polyketides and long-chain polyketides. For example, amphidinols are polyhydroxy-polyenes (long-chain polyketides) that have strong antifungal and haemolytic activity. They thus increase membrane permeability by associating with membrane lipids (6). Among the different Amphidinium strains, amphidinol-like compounds with a long polyhydroxy chain have been isolated, such as lingshuiols, karatungiols, carteraol E, luteophanols, colopsinols, and amphezonol A (5).

In order to limit the strong pressure of chemicals on the environment and health, it is necessary to isolate molecules of natural origin that will play a role in protecting crops against infectious diseases in order to eventually replace the chemical plant protection products used to date. These “organic pesticides” could thus directly affect the survival of phytopathogens of crops of major agronomic importance, such as wheat and grapevine.

Surprisingly, the inventors observed a fungicidal effect of a cell extract of Amphidinium carterae on many plant pathogenic fungi.

### SUMMARY OF THE INVENTION

A first object of the invention relates to the use of a cell extract of one or more microalgae of the genus Amphidinium for the fungicidal and/or bactericidal activity thereof on crop plant and seed pathogenic fungi, oomycetes and/or bacteria.

Another object of the invention relates to a process for preparing a cell extract of one or more microalga(e) of the genus Amphidinium characterized by the following steps:
Harvesting fresh cells from one or more microalga(e) of the genus Amphidinium
Optionally freezing and/or freeze-drying said cells
Suspending said fresh or frozen cells or said lyophilisate in an inorganic or organic solvent in a weight ratio of 1:200 to 1:2
Optionally freeze-drying the extract obtained.

Another object of the invention relates to a cell extract or lyophilisate of Amphidinium cells obtainable by the preceding process.

Another object of the invention relates to a process for controlling crop plant and seed pathogenic fungi, oomycetes and/or bacteria comprising applying to crop plants and/or coating said seeds with a cell extract of one or more microalgae of the genus Amphidinium or with an extract according to the invention.

Another object of the invention relates to a process for controlling crop plant and seed pathogenic fungi, oomycetes and/or bacteria, comprising the following steps:
Mixing extemporaneously in water, in a proportion of 1:4 to 1:800, a cell extract according to the invention
Applying this mixture to crop plants and/or coating said seeds with said mixture.

Another object of the invention relates to a process for controlling crop plant and seed pathogenic fungi, oomycetes and/or bacteria, comprising the following steps:
Resuspending a lyophilizate of a cell culture of one or more microalga(e) of the genus Amphidinium at a mass concentration comprised between 5 and 500 g/L, preferentially a mass concentration comprised between 50 and 400 g/L, preferentially a mass concentration comprised between 100 and 200 g/L, in water or in an organic or inorganic solvent in a weight ratio of 1:200 to 1:2,
Extemporaneously mixing in water, in a proportion of 1:4 to 1:800,
Applying the extract obtained to crop plants and/or coating said seeds with said mixture.

### FIGURE LEGENDS

FIG. 1. The Amphidinium carterae extract has an antifungal activity on Fusarium graminearum.

A. Fusarium graminearum spores were incubated in the presence of extracts from different microalgae cultures and then placed on in vitro culture medium. The photo was taken 72 hours later.

B. Fusarium graminearum spores were incubated in the presence of a range of concentrations of extracts (from 0 g/L to 2.0 g/L) from an Amphidinium carterae culture and then placed on in vitro culture medium. The photo was taken 72 hours later.

C. Similar to B but the number of germinated spores is counted 6 hours after incubation.

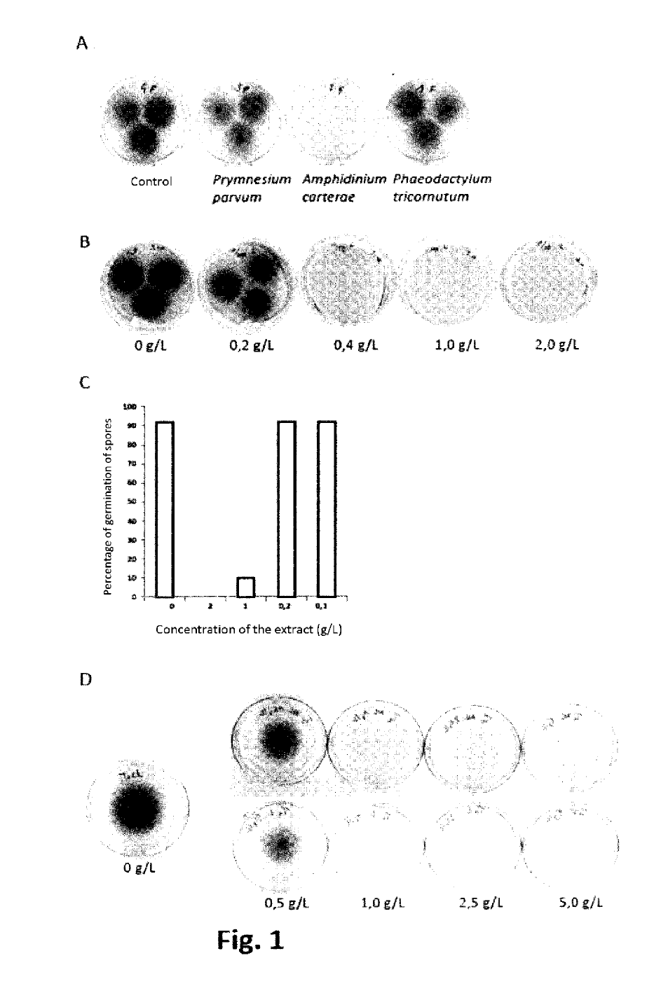

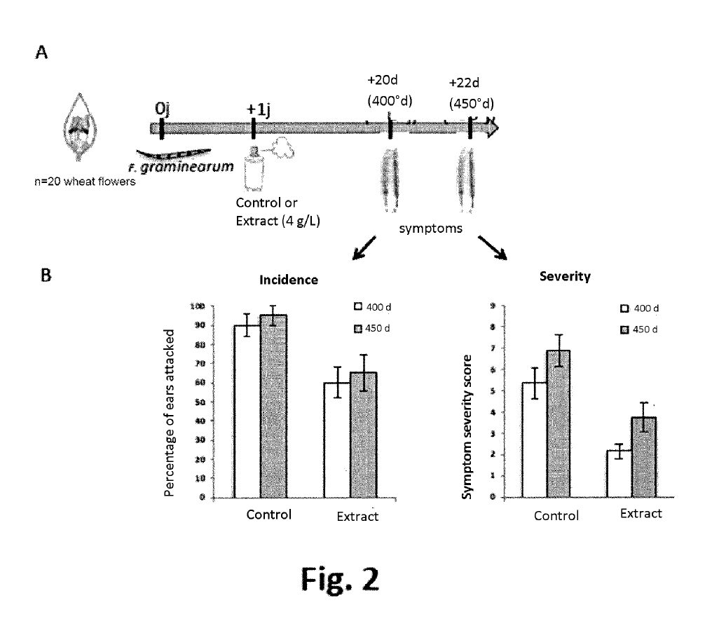

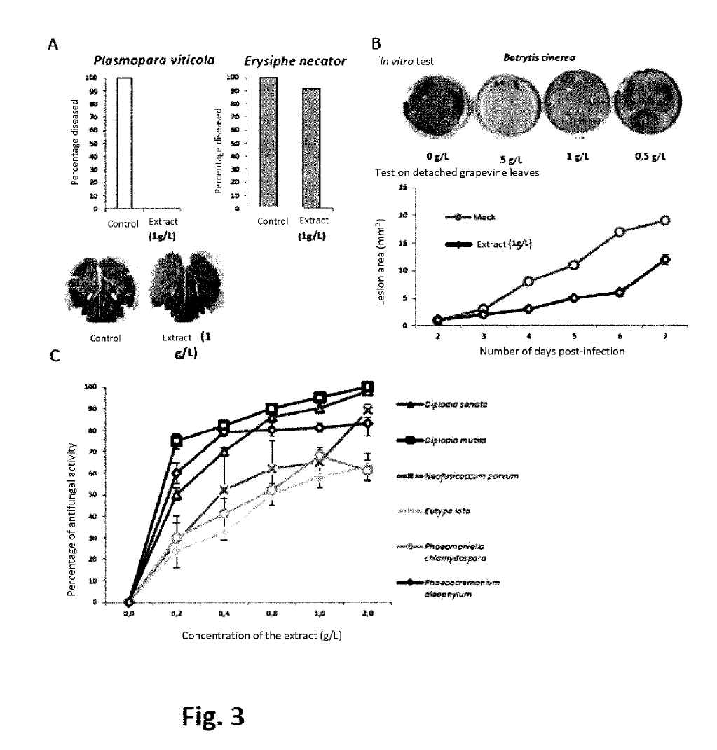

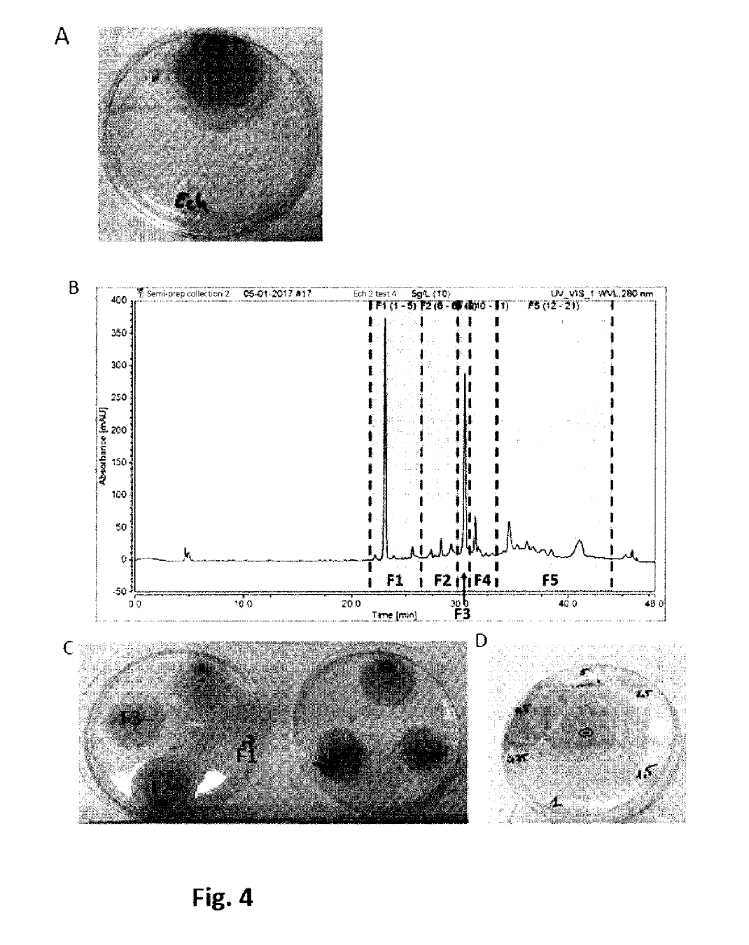

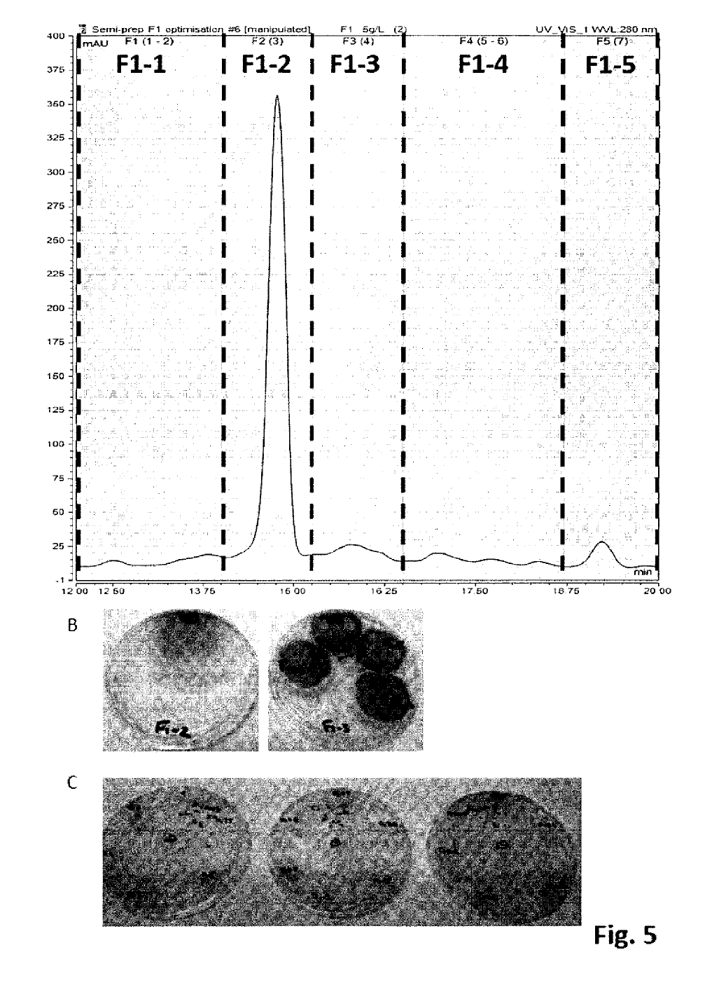

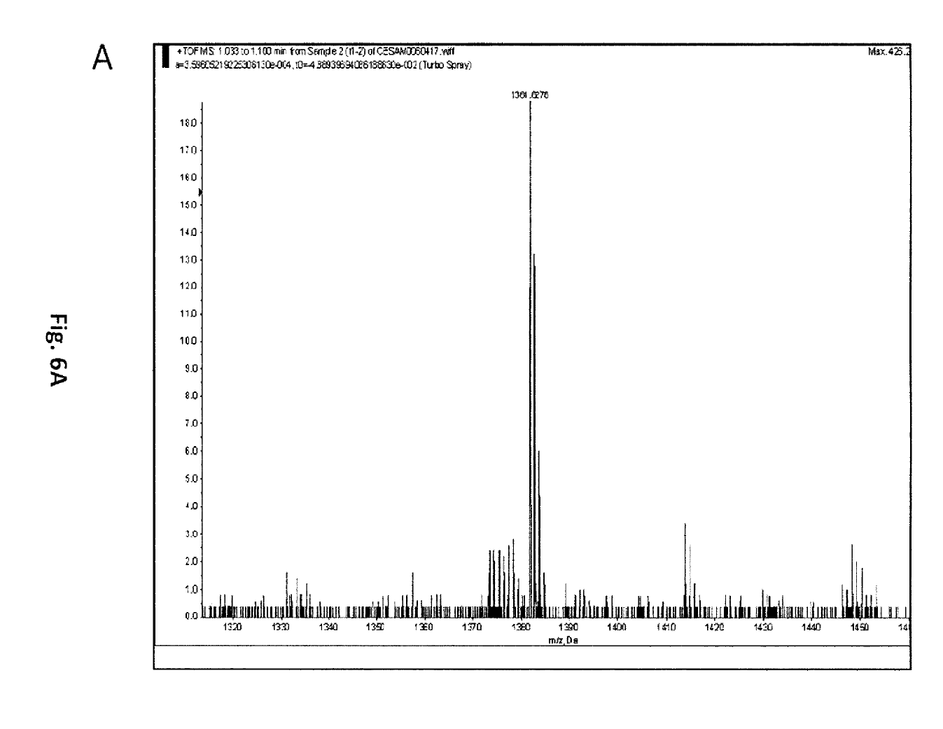

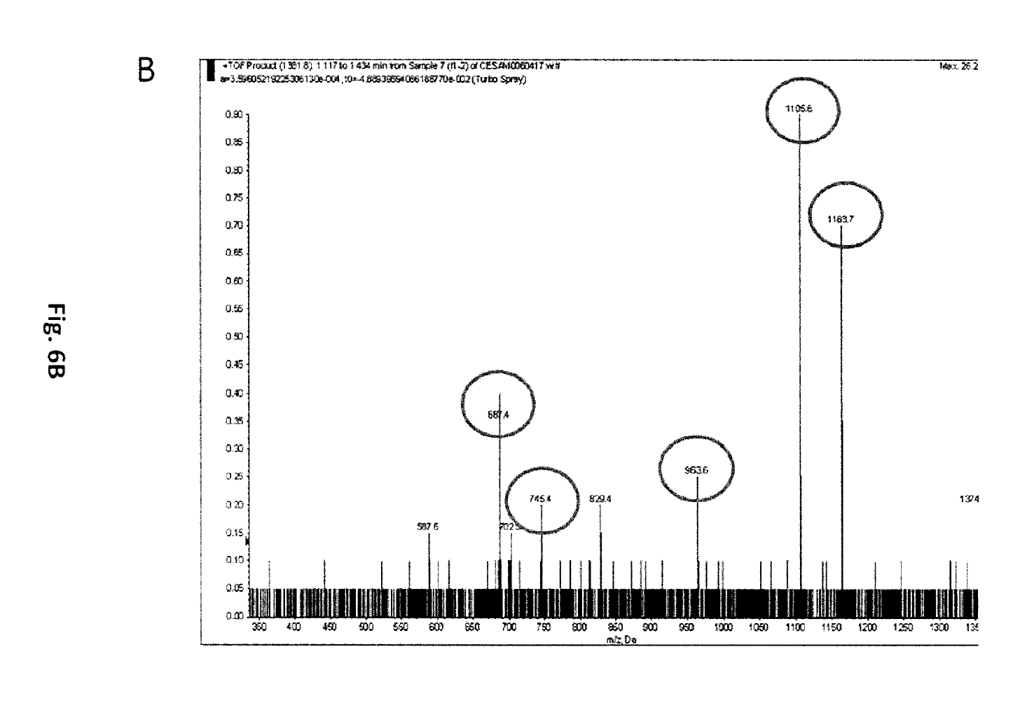

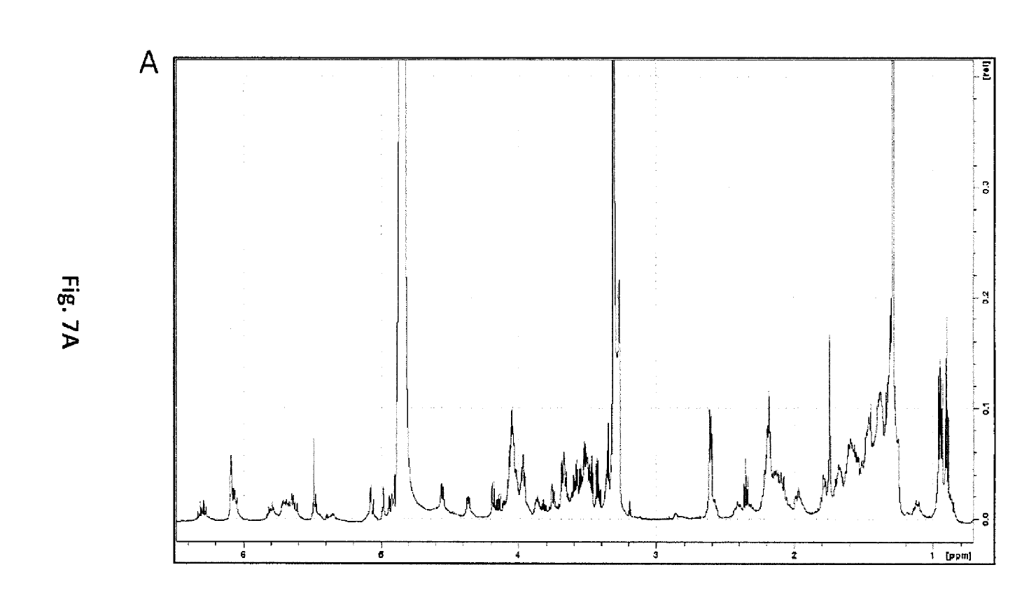

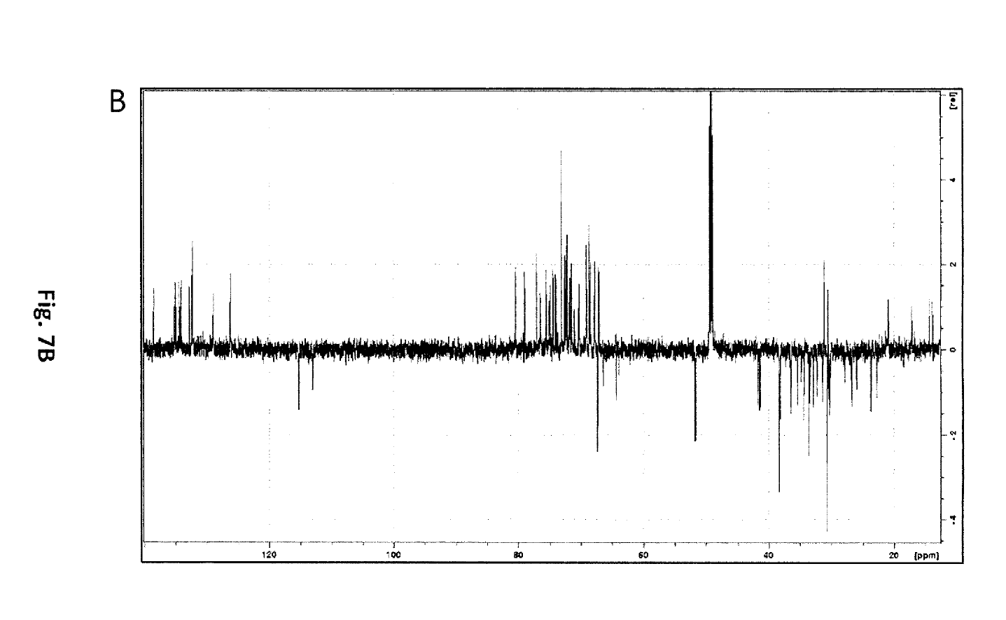

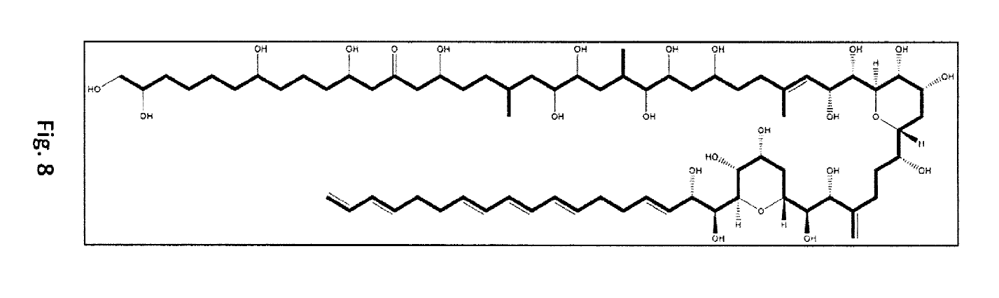

D. Fusarium graminearum spores were incubated in the presence of A. carterae extracts that were previously frozen (FIG. 1 D top row) or freeze-dried (FIG. 1 D bottom row).

FIG. 2. The Amphidinium carterae extract inhibits in vitro and in planta the growth of wheat pathogenic fungi.

A. Procedure for inoculating wheat flowers with Fusarium graminearum spores and treating them 24 hours later with a solution in the presence or absence (mock) of the Amphidinium carterae extract.

B. Symptom appearance and development are monitored and incidence and severity levels (score from 0 to 9) are recorded at 400° D and 450° D.

FIG. 3. The Amphidinium carterae extract inhibits in vitro and in planta the growth of grapevine pathogenic fungi

A. The Amphidinium carterae extract (1 g/L) or sterile water (mock) was sprayed on detached grapevine leaves maintained under in vitro conditions and then Plasmopara viticola sporangia or Erysiphe necator conidia were deposited on these leaves. Symptoms were read at 7 days and at 12 days, respectively.

B. Similar to A but Botrytis cinerea mycelial implants were deposited on the treated leaves. Symptoms were read at 7 days by measuring the size of the necrotic lesions.

C. Mycelial implants of different fungi involved in grapevine esca disease were deposited on a culture medium and treated 24 hours later with water (mock) or with A. carterae extract at various concentrations. Symptoms are read by measuring the surface area of the mycelium at 4 days.

FIG. 4: Fractionation of extract D on reversed-phase C18 column and activity test of the different fractions of extract D obtained by HPLC

A. In vitro test of F. graminearum growth inhibition by extract D at 5 g/L obtained after methanol extraction

B. Liquid chromatogram obtained after injection of extract D. The samples were assembled according to the dotted lines to form 5 fractions, denoted F1 to F5.

C. In vitro test of F. graminearum growth inhibition by fractions F1 to F5 at a concentration of 5 g/L

D. In vitro test of F. graminearum growth inhibition by fraction F1 at a concentration of 0.5 g/L, 0.75 g/L, 1 g/L, 1.5 g/L, 2.5 g/L, and 5 g/L

-: negative control: spores are incubated in the presence of the buffer alone

FIG. 5: Fractionation of fraction F1 on reversed-phase C18 column and activity test of the different fractions obtained by HPLC

A. Liquid chromatogram obtained after injection of fraction F1. The samples were assembled according to the dotted lines to form 5 fractions, denoted F1-1 to F1-5.

B. In vitro test of F. graminearum growth inhibition by fractions F1 to F5 at a concentration of 5 g/L

C. In vitro test of F. graminearum growth inhibition by fraction F1-2 at concentrations ranging from 0.0005 g/L to 1 g/L

-: negative control: spores are incubated in the presence of the buffer alone

FIG. 6. Mass spectrometry analysis of fraction F1-2.

A. Mass spectrum acquired by positive-mode electrospray ionization on the molecule of interest collected F1-2.

B. Tandem mass spectrum of the 1381.8276 Da ion detected in A. The circled peaks are consistent with those found in AM18 characterized by Nuzzo et al., 2014 (7).

FIG. 7. NMR analysis of fraction F1-2.

A. ¹H NMR spectrum (MeOD₄ solvent) obtained for the molecule of fraction F1-2 derived from extract D.

B. ¹³C DEPT135 NMR spectrum (MeOD₄ solvent) obtained for the molecule of fraction F1-2 derived from extract D.

FIG. 8. Structural formula of the amphidinol 18 molecule, derived from fraction F1-2 of extract D

All COSY and TOCSY correlations between each of the protons are shown in bold.

### DETAILED DESCRIPTION OF THE INVENTION

A first object of the invention relates to the use of a cell extract of one or more microalgae of the genus Amphidinium for the fungicidal and/or bactericidal activity thereof on crop plant and seed pathogenic fungi, oomycetes and/or bacteria.

Amphidinium Species

Suitable Amphidinium species are selected from the group consisting of Amphidinium achromaticum, Amphidinium aculeatum, Amphidinium acutissimum, Amphidinium acutum, Amphidinium alinii, Amphidinium aloxalocium, Amphidinium amphidinioides, Amphidinium asymmetricum, Amphidinium aureum, Amphidinium belauense, Amphidinium bidentatum, Amphidinium bipes, Amphidinium boekhoutensis, Amphidinium boggayum, Amphidinium caerulescens, Amphidinium carbunculus, Amphidinium carterae, Amphidinium celestinum, Amphidinium chattonii, Amphidinium coeruleum, Amphidinium conradii, Amphidinium conus, Amphidinium coprosum, Amphidinium corallinum, Amphidinium corpulentum, Amphidinium crassum, Amphidinium cristatum, Amphidinium cucurbita, Amphidinium cucurbitella, Amphidinium cupulatisquama, Amphidinium curvatum, Amphidinium cyaneoturbo, Amphidinium dentatum, Amphidinium discoidale, Amphidinium dubium, Amphidinium eilatiensis, Amphidinium emarginatum, Amphidinium fastigium, Amphidinium filum Bohm, Amphidinium flagellans, Amphidinium flexum, Amphidinium galbanum, Amphidinium gibbosum, Amphidinium glaucovirescens, Amphidinium glaucum, Amphidinium globosum, Amphidinium hadai, Amphidinium herdmanii, Amphidinium incoloratum, Amphidinium inflatum, Amphidinium kesselitzii, Amphidinium kesslitzii, Amphidinium klebsii, Amphidinium lacunarum, Amphidinium lanceolatum, Amphidinium lefevrei, Amphidinium lilloense, Amphidinium lissae, Amphidinium longum, Amphidinium luteum, Amphidinium machapungarum, Amphidinium macrocephalum, Amphidinium mammillatum, Amphidinium manannini, Amphidinium mananninii, Amphidinium massartii, Amphidinium mootonorum, Amphidinium mucicola, Amphidinium nasutum, Amphidinium obliquum, Amphidinium obrae, Amphidinium oceanicum, Amphidinium oculatum, Amphidinium operculatum, Amphidinium operculatum var. steinii, Amphidinium ornithocephalum, Amphidinium ovoideum, Amphidinium ovum, Amphidinium pacificum, Amphidinium pelagicum, Amphidinium phthartum, Amphidinium psammophila, Amphidinium psittacus, Amphidinium purpureum, Amphidinium pusillum, Amphidinium rhynchocephalum, Amphidinium roseolum, Amphidinium ruttneri, Amphidinium salinum, Amphidinium schilleri, Amphidinium schroederi, Amphidinium scissum, Amphidinium sphagnicola, Amphidinium sphenoides, Amphidinium steinii, Amphidinium stellatum, Amphidinium stigmatum, Amphidinium sulcatum, Amphidinium tortum, Amphidinium trochodinioides, Amphidinium trochodinoides, Amphidinium trulla, Amphidinium truncatum, Amphidinium turbo, Amphidinium vernal, Amphidinium vigrense, Amphidinium vitreum, Amphidinium vittatum, Amphidinium wigrense, Amphidinium yoorugurrum, Amphidinium yuroogurrum.

Preferably, the or one of the microalgae of the genus Amphidinium used according to the invention is Amphidinium carterae. There are several Amphidinium carterae strains in collection such as strains CCMP 124, 1314, 3177 (CCMP=Culture Collection of Marine Phytoplankton), AC 208, 792 (AC=Algobank Cean), BEA 01198 (BEA=Banco Espanol de Algas).

Advantageously, the Amphidinium carterae strain used according to the invention is CCMP 1314, AC208 or AC792.

Extraction

Said extract may be prepared by any cell extraction method known to the skilled person, solid-liquid or liquid-liquid, for example extraction in inorganic or organic solvent, which may be selected from the group consisting of water, aqueous solutions, ketones, esters, acids, ethers, alcohols and mixtures in any miscible proportions of these solvents.

Advantageously, the preferred solvent will be water or oxygenated solvents, preferably alcohols, particularly preferably C1 to C4 alcohols such as methanol or ethanol.

Particularly preferably, C1 to C4 alcohols are preferred.

Preferably, said extract is a water-soluble fraction.

Preferably, said extract comprises amphidinol 18 or amphidinol 19, particularly preferably amphidinol 18, advantageously in an amount greater than 1% w/w based on the total weight of the extract, preferably comprised between 2 and 10% w/w based on the total weight of the extract, particularly preferably comprised between 3 and 5% w/w based on the total weight of the extract.

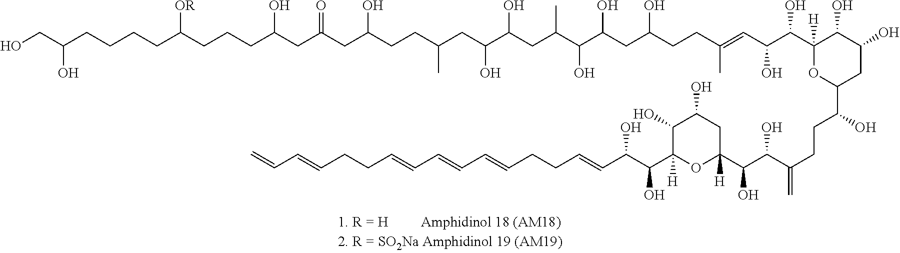

Mode of Action

Said fungicidal activity on crop plant and seed pathogenic fungi and/or oomycetes may be particularly exerted by inhibiting spore germination or by inhibiting fungal and/or oomycete growth.

The activity is exerted by cell wall and membrane lytic activity resulting in cell lysis.

Crop Plants

Said crop plants are in particular selected from the group consisting of cereals such as wheat, maize, barley, rice, soya, fruits and vegetables such as potato, carrot, apple trees, peach trees, apricot trees, tomatoes, radishes, beans, grapevine and ornamental plants.

Said crop plants are in particular selected from the group consisting of the genera Abelmoschus, Acacia, Achras, Agave, Agrostis, Aleurites, Allium, Anacardium, Ananas, Annona, Apium, Arachis, Areca, Armoracia, Arracacia, Artocarpus, Asparagus, Aspidosperma, Avena, Bertholletia, Beta, Boehmeria, Borassus, Brassica, Cajanus, Camellia, Cannabis, Capsicum, Carica, Carthamus, Carum, Carya, Castanea, Ceiba, Ceratonia, Chenopodium, Chrysanthemum, Cicer, Cichorium, Cinchona, Cinnamomum, Citrullus, Citrus, Cocos, Coffea, Cola, Colocasia, Corchorus, Corylus, Crotalaria, Cucumis, Cucurbita, Cydonia, Cymbopogon, Cynara, Dactylis, Daucus, Dioscorea, Diospyros, Echinochloa, Elaeis, Elettaria, Eleusine, Eragrostis, Eriobotrya, Eugenia, Fagopyrum, Ficus, Foeniculum, Fragaria, Furcraea, Glycine, Glycyrrhiza, Gossypium, Guizotia, Helianthus, Hevea, Hibiscus, Hordeum, Humulus, Ilex, Indigofera, Ipomoea, Jasminum, Juglans, Lactuca, Lagenaria, Lavandula, Lawsonia, Lens, Lepidium, Lespedeza, Linum, Litchi, Lolium, Lopmoea, Lotus, Lupinus, Lycopersicon, Lygeum, Macadamia, Malus, Mangifera, Manihot, Maranta, Medicago, Mentha, Mespilus, Metroxylon, Moringa, Musa, Myristica, Nicotiana, Olea, Onobrychis, Oryza, Panicum, Papaver, Pastinaca, Pelargonium, Pennisetum, Persea, Phaseolus, Phleum, Phoenix, Phormium, Pimpinella, Piper, Pistacia, Pisum, Prunus, Psidium, Punica, Pyrus, Raphanus Rheum, Ribes, Ricinus, Rose, Rubus, Saccharum, Scorzonera, Secale Sechium, Sesamum, Setaria, Solanum, Sorghum, Spinacia, Theobroma, Tragopogon, Trifolium, Trigonefia, Triticum, Urena, Vaccinium, Valerianella, Vanilla, Vicia, Vigna, Vitellaria, Vitis, Xanthosoma, Zea, Zingiber.

Pathogens

Said crop plant and seed pathogenic fungi are ascomycetes or basidiomycetes, preferably ascomycetes.

Said crop plant and seed pathogenic fungi are fungi pathogenic to crop plants and seeds of the genera:

Acrocalymma, Acrocalymma medicaginis,

Fusarium, Fusarium affine, Fusarium arthrosporioides, Fusarium crookwefiense, Fusarium culmorum, Fusarium graminearum, Fusarium monihforme, Fusarium incarnatum, Fusarium solani, Fusarium langsethiae, Fusarium mangiferae, Fusarium oxysporum f. sp. albedinis, Fusarium oxysporum f. sp. asparagi, Fusarium oxysporum f. sp. batatas, Fusarium oxysporum f. sp. betae, Fusarium oxysporum f. sp. cannabis, Fusarium oxysporum f. sp. carthami, Fusarium oxysporum f. sp. cattleyae, Fusarium oxysporum f. sp. ciceris, Fusarium oxysporum f. sp. coffea, Fusarium oxysporum f. sp. cubense, Fusarium oxysporum f. sp. cyclaminis, Fusarium oxysporum f. sp. dianthi, Fusarium oxysporum f. sp. lentis, Fusarium oxysporum f. sp. lini, Fusarium oxysporum f. sp. lycopersici, Fusarium oxysporum f. sp. medicaginis, Fusarium oxysporum f. sp. pisi, Fusarium oxysporum f. sp. radicis-lycopersici, Fusarium oxysporum f. sp. spinacia, Fusarium oxysporum, Fusarium pallidoroseum, Fusarium patch, Fusarium proliferatum, Fusarium redolens, Fusarium sacchari, Fusarium solani, Fusarium subglutinans, Fusarium sulphureum, Fusarium tricinctum, Fusarium wilt,

Botrytis, Botrytis allii, Botrytis anthophila, Botrytis cinerea, Botrytis fabae, Botrytis narcissicola,

Alternaria, Alternaria alternata, Alternaria brassicae, Alternaria brassicicola, Alternaria carthami, Alternaria cinerariae, Alternaria dauci, Alternaria dianthi, Alternaria dianthicola, Alternaria euphorbiicola, Alternaria helianthi, Alternaria helianthicola, Alternaria japonica, Alternaria leucanthemi, Alternaria limicola, Alternaria linicola, Alternaria padwickii, Alternaria panax, Alternaria radicina, Alternaria raphani, Alternaria saponariae, Alternaria senecionis, Alternaria solani, Alternaria tenuissima, Alternaria triticina, Alternaria zinniae,

Erisyphe, Erisyphe necator, Erysiphe betae, Erysiphe brunneopunctata, Erysiphe cichoracearum, Erysiphe cruciferarum, Erysiphe graminis f. sp. Avenae, Erysiphe graminis f. sp. tritici, Erysiphe heraclei, Erysiphe pisi,

Claviceps, Claviceps fusiformis, Claviceps purpurea, Claviceps sorghi, Claviceps zizaniae,

Gaeumannomyces, Gaeumannomyces graminis,

Leptosphaeria, Leptosphaeria nodorum, Leptosphaeria acuta, Leptosphaeria cannabina, Leptosphaeria coniothyrium, Leptosphaeria libanotis, Leptosphaeria lindquistii, Leptosphaeria maculans, Leptosphaeria musarum, Leptosphaeria pratensis, Leptosphaeria sacchari, Leptosphaeria woroninii,

Microdochium, Microdochium spp. Microdochium bolleyi, Microdochium dimerum, Microdochium panattonianum, Microdochium phragmitis,

Mycosphaerella, Mycosphaerella arachidis, Mycosphaerella areola, Mycosphaerella berkeleyi, Mycosphaerella bolleana, Mycosphaerella brassicicola, Mycosphaerella caricae, Mycosphaerella caryigena, Mycosphaerella cerasella, Mycosphaerella coffeicola, Mycosphaerella confusa, Mycosphaerella cruenta, Mycosphaerella dendroides, Mycosphaerella eumusae, Mycosphaerella gossypina, Mycosphaerella graminicola, Mycosphaerella henningsii, Mycosphaerella horii, Mycosphaerella juglandis, Mycosphaerella lageniformis, Mycosphaerella linicola, Mycosphaerella louisianae, Mycosphaerella musae, Mycosphaerella musicola, Mycosphaerella palmicola, Mycosphaerella pinodes, Mycosphaerella pistaciarum, Mycosphaerella pistacina, Mycosphaerella platanifolia, Mycosphaerella polymorpha, Mycosphaerella pomi, Mycosphaerella punctiformis, Mycosphaerella pyri,

Oculimacula, Oculimacula acuformis, Oculimacula yallundae,

Blumeria, Blumeria graminis,

Pyrenophora, Pyrenophora avenae, Pyrenophora chaetomioides, Pyrenophora graminea, Pyrenophora seminiperda, Pyrenophora teres, Pyrenophora teres f. maculata, Pyrenophora teres f. teres, Pyrenophora triticirepentis,

Ramularia, Ramularia collocygni, Ramularia beticola, Ramularia coryli, Ramularia cyclaminicola, Ramularia macrospora, Ramularia menthicola, Ramularia necator, Ramularia primulae, Ramularia spinaciae, Ramularia subtilis, Ramularia tenella, Ramularia yallisumbrosae,

Rhynchosporium, Rhynchosporium secalis,

Cochliobolus, Cochliobolus, Cochliobolus carbonum, Cochliobolus cymbopogonis, Cochliobolus hawaiiensis, Cochliobolus heterostrophus, Cochliobolus lunatus, Cochliobolus miyabeanus, Cochliobolus rayenelii, Cochliobolus sativus, Cochliobolus setariae, Cochliobolus spicifer, Cochliobolus stenospilus, Cochliobolus tuberculatus, Cochliobolus victoriae,

Microdochium, Microdochium oryzae,

Pyricularia, Pyricularia oryzae,

Sarocladium, Sarocladium oryzae,

Ustilaginoides, Ustilaginoides virens,

Cercospora, Cercospora, Cercospora apii, Cercospora apii f. sp. clerodendri, Cercospora apiicola, Cercospora arachidicola, Cercospora asparagi, Cercospora atrofiliformis, Cercospora beticola, Cercospora brachypus, Cercospora brassicicola, Cercospora brunkii, Cercospora cannabis, Cercospora cantuariensis, Cercospora capsici, Cercospora carotae, Cercospora corylina, Cercospora fuchsiae, Cercospora fusca, Cercospora fusimaculans, Cercospora gerberae, Cercospora halstedii, Cercospora handelii, Cercospora hayi, Cercospora hydrangeae, Cercospora kikuchii, Cercospora lentis, Cercospora liquidambaris, Cercospora longipes, Cercospora longissima, Cercospora mamaonis, Cercospora mangiferae, Cercospora medicaginis, Cercospora melongenae, Cercospora minuta, Cercospora nicotianae, Cercospora odontoglossi, Cercospora papayae, Cercospora penniseti, Cercospora pisa sativae, Cercospora platanicola, Cercospora puderii, Cercospora pulcherrima, Cercospora rhapidicola, Cercospora rosicola, Cercospora sojina, Cercospora solani, Cercospora solanituberosi, Cercospora sorghi, Cercospora theae, Cercospora tuberculans, Cercospora vexans, Cercospora vicosae, Cercospora zeaemaydis, Cercospora zebrina, Cercospora zonata,

Corynespora, Corynespora cassiicola,

Phakospora, Phakospora pachyrhizi, Phakopsora gossypii, Colletotrichum, Colletotrichum acutatum, Colletotrichum arachidis, Colletotrichum capsici, Colletotrichum cereale, Colletotrichum coffeanum, Colletotrichum crassipes, Colletotrichum dematium, Colletotrichum dematium f. spinaciae, Colletotrichum derridis, Colletotrichum destructivum, Colletotrichum gloeosporioides, Colletotrichum glycines, Colletotrichum gossypii, Colletotrichum graminicola, Colletotrichum higginsianum, Colletotrichum kahawae, Colletotrichum lindemuthianum, Colletotrichum lini, Colletotrichum mangenotii, Colletotrichum musae, Colletotrichum nigrum, Colletotrichum orbiculare, Colletotrichum pisi, Colletotrichum sublineolum, Colletotrichum trichellum, Colletotrichum trifolii, Colletotrichum truncatum,

Pythium spp.,

Diplodia, Diplodia allocellula, Diplodia laeliocattleyae, Diplodia manihoti, Diplodia paraphysaria, Diplodia seriata, Diplodia theae-sinensis,

Monilia, Monilinia azaleae, Monilinia fructicola, Monilinia fructigena, Monilinia laxa, Monilinia oxycocci,

Pezzicula, Pezzicula alba, Pezzicula malicorticis,

Zymoseptoria, Zymoseptoria tritici

Phytophthora, Phytophthora infestans

Guignardia, Guignardia bidwelli, Guignardia camelliae, Guignardia fulvida, Guignardia mangiferae, Guignardia musae, Guignardia philoprina,

Plasmopara, Plasmopara viticola,

Puccinia, Puccinia angustata, Puccinia arachidis, Puccinia aristidae, Puccinia asparagi, Puccinia cacabata, Puccinia campanulae, Puccinia carthami, Puccinia coronata, Puccinia dioicae, Puccinia erianthi, Puccinia extensicola, Puccinia helianthi, Puccinia hordei, Puccinia jaceae, Puccinia kuehnii, Puccinia malvacearum, Puccinia mariae-wilsoniae, Puccinia melanocephala, Puccinia menthae, Puccinia oxalidis, Puccinia pelargonii-zonalis, Puccinia pittieriana, Puccinia poarum, Puccinia purpurea, Puccinia recondita, Puccinia schedonnardii, Puccinia sessilis, Puccinia striiformis, Puccinia striiformis, Puccinia subnitens, Puccinia substriata, Puccinia verruca, Puccinia xanthii,

Rhizoctonia, Rhizoctonia solani, Rhizoctonia oryzae, Rhizoctonia cerealis, Rhizoctonia leguminicola, Rhizoctonia rubi,

Sclerotinia, Sclerotinia borealis, Sclerotinia bulborum, Sclerotinia minor, Sclerotinia ricini, Sclerotinia sclerotiorum, Sclerotinia spermophila, Sclerotinia trifoliorum,

Septoria, Septoria ampelina, Septoria azaleae, Septoria bataticola, Septoria campanulae, Septoria cannabis, Septoria cucurbitacearum, Septoria darrowii, Septoria dianthi, Septoria eumusae, Septoria glycines, Septoria helianthi, Septoria humuli, Septoria hydrangeae, Septoria lactucae, Septoria lycopersici, Septoria lycopersici, Septoria menthae, Septoria passerinii, Septoria pisi, Septoria rhododendri, Septoria secalis, Septoria selenophomoides,

Venturia, Venturia inaequalis. Venturia carpophila,

Acrodontium, Acrodontium simplex,

Acrophialophora, Acrophialophora fusispora,

Acrosporium, Acrosporium tingitaninum,

Aecidium, Aecidium aechmantherae, Aecidium amaryllidis, Aecidium breyniae, Aecidium campanulastri, Aecidium cannabis, Aecidium cantensis, Aecidium caspicum, Aecidium foeniculi, Aecidium narcissi,

Ahmadiago,

Albonectria, Albonectria rigidiuscula,

Allodus, Allodus podophylli,

Amphobotrys, Amphobotrys ricini,

Anguillosporella, Anguillosporella vermiformis,

Anthostomella, Anthostomella pullulans,

Antrodia, Antrodia albida, Antrodia serialiformis, Antrodia serialis,

Apiospora, Apiospora montagnei,

Appendiculella,

Armillaria Armillaria heimii, Armillaria sinapina, Armillaria socialis, Armillaria tabescens,

Arthrocladiella,

Arthuriomyces, Arthuriomyces peckianus,

Ascochyta, Ascochyta asparagina, Ascochyta bohemica, Ascochyta caricae, Ascochyta doronici, Ascochyta fabae f. sp. lentis, Ascochyta graminea, Ascochyta hordei, Ascochyta humuli, Ascochyta pisi, Ascochyta prasadii, Ascochyta sorghi, Ascochyta spinaciae, Ascochyta tarda, Ascochyta tritici,

Ascospora, Ascospora ruborum,

Aspergillus, Aspergillus aculeatus, Aspergillus fischerianus, Aspergillus niger,

Asperisporium, Asperisporium caricae,

Asteridiella,

Asteroma, Asteroma caryae,

Athelia, Athelia arachnoidea, Athelia rolfsii,

Aurantiporus, Aurantiporus fissilis,

Aureobasidium, Aureobasidium pullulans,

Bambusiomyces,

Banana freckle,

Bayoud disease,

Beniowskia, Beniowskia sphaeroidea,

Bionectria, Bionectria ochroleuca,

Bipolaris, Bipolaris cactivora, Bipolaris cookei, Bipolaris incurvata, Bipolaris sacchari,

Biscogniauxia, Biscogniauxia capnodes, Biscogniauxia marginata,

Bjerkandera, Bjerkandera adusta,

Black sigatoka,

Blakeslea, Blakeslea trispora,

Botryodiplodia, Botryodiplodia oncidii, Botryodiplodia ulmicola,

Botryosphaeria, Botryosphaeria cocogena, Botryosphaeria dothidea, Botryosphaeria marconii, Botryosphaeria obtusa, Botryosphaeria rhodina, Botryosphaeria ribis, Botryosphaeria stevensii,

Botryosporium, Botryosporium pulchrum,

Botryotinia, Botryotinia fuceliana, Botryotinia polyblastis,

Boxwood blight,

Brachybasidiaceae,

Brasiliomyces, Brasiliomyces malachrae,

Briosia, Briosia ampelophaga,

Brown ring patch,

Buckeye rot of tomato,

Bulbomicrosphaera,

Cadophora, Cadophora malorum,

Caespitotheca,

Calonectria, Calonectria ilicicola, Calonectria indusiata, Calonectria kyotensis, Calonectria pyrochroa, Calonectria quinqueseptata,

Camarotella, Camarotella acrocomiae, Camarotella costaricensis,

Canna rust,

Capitorostrum, Capitorostrum cocoes,

Capnodium, Capnodium footii, Capnodium mangiferum, Capnodium ramosum, Capnodium theae,

Cephalosporium, Cephalosporium gramineum,

Ceratobasidium, Ceratobasidium cereale, Ceratobasidium cornigerum, Ceratobasidium noxium, Ceratobasidium ramicola, Ceratobasidium setariae, Ceratobasidium stevensii,

Ceratocystis, Ceratocystis adiposa, Ceratocystis coerulescens, Ceratocystis fimbriata, Ceratocystis moniliformis, Ceratocystis oblonga, Ceratocystis obpyriformis, Ceratocystis paradoxa, Ceratocystis pilifera, Ceratocystis pluriannulata, Ceratocystis polyconidia, Ceratocystis tanganyicensis, Ceratocystis zombamontana,

Ceratorhiza, Ceratorhiza hydrophila,

Ceratospermopsis,

Cercoseptoria, Cercoseptoria ocellata,

Cercosporella, Cercosporella rubi,

Ceriporia, Ceriporia spissa, Ceriporia xylostromatoides,

Cerrena, Cerrena unicolor,

Ceuthospora, Ceuthospora lauri,

Choanephora, Choanephora cucurbitarum, Choanephora infundibulifera,

Chrysanthemum, Chrysanthemum white rust,

Chrysomyxa, Chrysomyxa cassandrae,

Chrysomyxa, Chrysomyxa himalensis, Chrysomyxa ledi, Chrysomyxa ledi var. rhododendri, Chrysomyxa ledicola, Chrysomyxa nagodhii, Chrysomyxa neoglandulosi, Chrysomyxa piperiana, Chrysomyxa pirolata, Chrysomyxa pyrolae, Chrysomyxa reticulata, Chrysomyxa roanensis, Chrysomyxa succinea,

Cladosporium, Cladosporium arthropodii, Cladosporium cladosporioides, Cladosporium cladosporioides f. sp. pisicola, Cladosporium cucumerinum, Cladosporium herbarum, Cladosporium musae, Cladosporium oncobae,

Climacodon, Climacodon pulcherrimus, Climacodon septentrionalis,

Clitocybe, Clitocybe parasitica,

Clonostachys rosea f. rosea,

Clypeoporthe, Clypeoporthe iliau,

Coleosporium, Coleosporium helianthi, Coleosporium ipomoeae, Coleosporium madiae, Coleosporium pacificum, Coleosporium tussilaginis,

Conidiosporomyces,

Coniella, Coniella castaneicola, Coniella diplodiella, Coniella fragariae,

Coniothecium, Coniothecium chomatosporum,

Coniothyrium, Coniothyrium celtidisaustralis, Coniothyrium henriquesii, Coniothyrium rosarum, Coniothyrium wernsdorffiae,

Coprinopsis, Coprinopsis psychromorbida, Cordana, Cordana johnstonii, Cordana musae, Coriolopsis floccosa,

Corn grey leaf spot,

Corticium, Corticium invisum, Corticium penicillatum, Corticium theae,

Coryneopsis, Coryneopsis rubi,

Coryneum, Coryneum rhododendri,

Covered smut,

Crinipellis, Crinipellis sarmentosa,

Cronartium, Cronartium ribicola,

Cryphonectriaceae,

Cryptobasidiaceae,

Cryptocline, Cryptocline cyclaminis,

Cryptomeliola,

Cryptosporella, Cryptosporella umbrina,

Cryptosporiopsis, Cryptosporiopsis tarraconensis,

Cryptosporium, Cryptosporium minimum,

Curvularia, Curvularia lunata, Curvularia caricaepapayae, Curvularia penniseti, Curvularia senegalensis, Curvularia trifolii,

Cyclaneusma needle cast,

Cylindrocarpon, Cylindrocarpon ianthothele var. ianthothele, Cylindrocarpon magnusianum, Cylindrocarpon musae,

Cylindrocladiella, Cylindrocladiella camelliae, Cylindrocladiella parva,

Cylindrocladium, Cylindrocladium clavatum, Cylindrocladium lanceolatum, Cylindrocladium peruvianum, Cylindrocladium pteridis,

Cylindrosporium, Cylindrosporium cannabinum, Cylindrosporium juglandis, Cylindrosporium rubi,

Cymadothea, Cymadothea trifolii,

Cytospora, Cytospora palmarum, Cytospora personata, Cytospora sacchari, Cytospora sacculus, Cytospora terebinthi,

Cytosporina, Cytosporina ludibunda,

Dactuliophora, Dactuliophora elongata,

Davidiella, Davidiella dianthi, Davidiella tassiana,

Deightoniella, Deightoniella papuana, Deightoniella torulosa,

Dendrophora, Dendrophora marconii, Dendrophora erumpens,

Denticularia, Denticularia mangiferae,

Dermea pseudotsugae,

Diaporthaceae,

Diaporthe, Diaporthe arctii, Diaporthe dulcamarae, Diaporthe eres, Diaporthe helianthi, Diaporthe lagunensis, Diaporthe lokoyae, Diaporthe melonis, Diaporthe orthoceras, Diaporthe perniciosa, Diaporthe phaseolorum, Diaporthe phaseolorum var. caulivora, Diaporthe phaseolorum var. phaseolorum, Diaporthe phaseolorum var. soja, Diaporthe rudis, Diaporthe tanakae, Diaporthe toxica,

Dicarpella, Dicarpella dryina,

Didymella, Didymella applanata, Didymella bryoniae, Didymella fabae, Didymella lycopersici

Didymosphaeria, Didymosphaeria arachidicola, Didymosphaeria taiwanensis,

Dilophospora, Dilophospora alopecuri,

Dimeriella, Dimeriella sacchari,

Diplocarpon, Diplocarpon mespili, Diplocarpon rosae,

Discosia, Discosia artocreas,

Discostroma, Discostroma corticola,

Distocercospora, Distocercospora livistonae,

Dothiorella, Dothiorella brevicollis, Dothiorella dominicana, Dothiorella dulcispinae, Dothiorella gregaria,

Drechslera, Drechslera avenacea, Drechslera campanulata, Drechslera dematioidea, Drechslera gigantea, Drechslera glycines, Drechslera musaesapientium, Drechslera teres f. maculata, Drechslera wirreganensis,

Eballistra, Eballistra lineata, Eballistra oryzae,

Eballistraceae,

Echinodondum, Echinodontium ryvardenii, Echinodontium tinctorium,

Ectendomeliola,

Elsinoë, Elsinoë ampelina, Elsinoë batatas, Elsinoë brasiliensis, Elsinoë leucospila, Elsinoë randii, Elsinoë rosarum, Elsinoë sacchari, Elsinoë theae, Elsinoë veneta,

Endomeliolo,

Endothia, Endothia radicalis,

Endothiella, Endothiella gyrosa,

Entorrhizomycetes,

Entyloma, Entyloma ageratinae, Entyloma dahliae, Entyloma ellisii,

Epicoccum, Epicoccum nigrum,

Eremothecium, Eremothecium coryli, Eremothecium gossypii,

Erysipholes,

Exobasidiaceae, Exobasidium burtii, Exobasidium reticulatum, Exobasidium yaccinii var. japonicum,

Exobasidium vaccinii-uliginosi, Exobasidium vexans, xxophiala alcalophila,

Exophiala, Exophiala angulospora, Exophiala attenuata, Exophiala calicioides, Exophiala costellanii, Exophiala dermatitidis, Exophiala dopicolo, Exophiala exophialae, Exophiala heteromorpha, Exophiala hongkongensis, Exophiala jeanselmei, Exophiala lecaniicorni, Exophiala monsonii, Exophiala mesophila, Exophiala moniliae, Exophiala negronii, Exophiala phaeomuriformis, Exophiala pisciphila, Exophiala psychrophila, Exophiala salmonis, Exophiala spinifera,

Fomes, Fomes lamaensis,

Fomitopsis, Fomitopsis rosea,

Fusicladium pisicolo,

Fusicoccum, Fusicoccum oesculi, Fusicoccum amygdali, Fusicoccum quercus,

Galactomyces, Galactomyces candidum,

Ganoderma, Ganoderma brownii, Ganoderma lobatum, Ganoderma megaloma, Ganoderma meredithiae, Ganoderma orbiforme, Ganoderma philippii, Ganoderma sessile, Ganoderma tornatum, Ganoderma zonatum,

Geastrumia, Geastrumia polystigmatis,

Georgefischeriaceae,

Georgefischeriales,

Geosmithia, Geosmithia pallida,

Geotrichum, Geotrichum candidum, Geotrichum klebahnii,

Gibberella, Gibberella acuminata, Gibberella avenacea, Gibberella baccata, Gibberella cyanogena, Gibberella fujikuroi, Gibberella intricans, Gibberella pulicaris, Gibberella stilboides, Gibberella tricincta, Gibberella xylarioides, Gibberella zeae,

Gibellina, Gibellina cerealis,

Gilbertella, Gilbertella persicaria,

Gjaerumiaceae,

Gliocladiopsis, Gliocladiopsis tenuis,

Gliocladium, Gliocladium vermoeseni,

Gloeocercospora, Gloeocercospora sorghi,

Gloeocystidiellum, Gloeocystidiellum porosum,

Gloeophyllum, Gloeophyllum mexicanum, Gloeophyllum trabeum,

Gloeoporus, Gloeoporus dichrous,

Gloeosporium, Gloeosporium cattleyae, Gloeosporium theae-sinensis,

Glomerella, Glomerella cingulata, Glomerella graminicola, Glomerella tucumanensis,

Gnomonia, Gnomonic caryae, Gnomonic comari, Gnomonic dispora, Gnomonic iliau, Gnomonic rubi,

Golovinomyces, Golovinomyces cichoracearum,

Graphiola phoenicis,

Graphiolaceae,

Graphium, Graphium rigidum, Graphium rubrum,

Graphyllium, Graphyllium pentamerum,

Grovesinia, Grovesinia pyramidalis,

Gymnoconia, Gymnoconia nitens,

Gymnopus, Gymnopus dryophilus,

Gymnosporangium, Gymnosporangium kernianum, Gymnosporangium libocedri, Gymnosporangium nelsonii, Gymnosporangium yamadae,

Haematonectria, Haematonectria haematococca,

Hansenula, Hansenula subpelliculosa,

Hapalosphaeria, Hapalosphaeria deformans,

Haplobasidion, Haplobasidion musae,

Helicobasidium, Helicobasidium compactum, Helicobasidium longisporum, Helicobasidium purpureum,

Helicoma, Helicoma muelleri,

Helminthosporium, Helminthosporium cookei, Helminthosporium solani,

Hendersonia, Hendersonia creberrima, Hendersonia theicola,

Hericium, Hericium coralloides,

Heterobasidion, Heterobasidion irregulare, Heterobasidion occidentale,

Hexagonia, Hexagonia hydnoides,

Hymenula, Hymenula affinis,

Hyphodermella, Hyphodermella corrugata,

Hyphodontia, Hyphodontia aspera, Hyphodontia sambuci,

Hypoxylon, Hypoxylon tinctor,

Inonotus, Inonotus arizonicus, Inonotus cuticularis, Inonotus dryophilus, Inonotus hispidus, Inonotus ludoyicianus,

Irpex, Irpex destruens, Irpex lacteus,

Kabatiella, Kabatiella couliyora,

Kornal bunt,

Koa wilt,

Kretzschmaria, Kretzschmaria zonata,

Kuehneola, Kuehneola uredinis,

Kutilakesa, Kutilakesa pironii,

Laetiporus, Laetiporus ailaoshanensis, Laetiporus baudonii, Laetiporus caribensis, Laetiporus conifericola, Laetiporus cremeiporus, Laetiporus gilbertsonii, Laetiporus huroniensis, Laetiporus montanus, Laetiporus portentosus, Laetiporus zonatus,

Laxitextum, Laxitextum bicolor,

Leandria, Leandria momordicae,

Lentinus, Lentinus tigrinus,

Lenzites, Lenzites betulina, Lenzites elegans,

Leohumicola, Leohumicola atra, Leohumicola incrustata, Leohumicola levissima,

Leptodontidium, Leptodontidium elatius,

Leptographium, Leptographium microsporum,

Leptosphaerulina, Leptosphaerulina crassiasca, Leptosphaerulina trifolii,

Leptothyrium, Leptothyrium nervisedum,

Leptotrochila, Leptotrochila medicaginis,

Leucocytospora, Leucocytospora leucostoma,

Leucostoma, Leucostoma auerswaldii, Leucostoma canker, Leucostoma kunzei, Leucostoma persoonii,

Leveillula, LevelHula compositarum, Leveillula leguminosarum, Leveillula taurica,

Limacinula, Limacinula tenuis,

Linochora, Linochora graminis,

Loose smut,

Lopharia, Lopharia crassa,

Lophodermium, Lophodermium aucupariae, Lophodermium schweinitzii,

Macrophoma, Macrophoma mangiferae, Macrophoma theicola,

Macrosporium, Macrosporium cocos,

Magnaporthe, Magnaporthe grisea, Magnaporthe salvia,

Magnaporthiopsis,

Mamianiella, Mamianiella coryli,

Marasmiellus, Marasmiellus cocophilus, Marasmiellus stenophyllus,

Marasmius, Marasmius crinisequi, Marasmius sacchari, Marasmius semiustus, Marasmius stenophyllus, Marasmius tenuissimus,

Massarina, Massarina walkeri,

Mauginiella, Mauginiella scaettae,

Melampsora, Melampsora lini, Melampsora occidentalis,

Melanconis, Melanconis carthusiana,

Melanconium, Melanconium juglandinum,

Meliola, Mellola mangiferae, Meliola zangii,

Meruliopsis, Meruliopsis ambigua,

Microascus, Microascus breyicaulis,

Microbotryum, Microbotryum silenesdioicae, Microbotryum violaceum,

Microsphaera, Microsphaera coryli, Microsphaera diffusa, Microsphaera ellisii, Microsphaera euphorbiae, Microsphaera hommae, Microsphaera penicillata, Microsphaera yaccinii, Microsphaera verruculosa,

Microstroma, Microstroma juglandis,

Moesziomyces, Moesziomyces bullatus,

Moniliophthora, Moniliophthora roreri,

Monilochaetes, Monilochaetes infuscans,

Monochaetia, Monochaetia coryli, Monochaetia mall,

Monographella, Monographella albescens, Monographella cucumerina, Monographella niyalis,

Monosporascus, Monosporascus cannonballus, Monosporascus eutypoides,

Monostichella, Monostichella coryli,

Mucor, Mucor circinelloides, Mucor hiemalis, Mucor mucedo, Mucor paronychius, Mucor piriformis, Mucor racemosus,

Mycena, Mycena citricolor,

Mycocentrospora, Mycocentrospora acerina,

Mycoleptodiscus, Mycoleptodiscus terrestris,

Didymella, Didymella rabiei,

Mycosphaerella, Mycosphaerella recutita, Mycosphaerella rosicola, Mycosphaerella rubi, Mycosphaerella stigminaplatani, Mycosphaerella striatiformans,

Mycovellosiella, Mycovellosiella concors,

Passalora, Passalora fulva,

Mycovellosiella, Mycovellosiella koepkei, Mycovellosiella vaginae,

Myriogenospora, Myriogenospora aciculispora,

Myrothecium, Myrothecium roridum, Myrothecium verrucaria,

Naevala, Naevala perexigua,

Naohidemyces, Naohidemyces vaccinii,

Nectria, Nectria cinnabarina, Nectria ditissima, Nectria foliicola, Nectria mammoidea, Nectria mauritiicola, Nectria peziza, Nectria pseudotrichia, Nectria radicicola, Nectria ramulariae,

Nectriella, Nectriella pironii,

Nemania, Nemania diffusa, Nemania serpens,

Neocosmospora, Neocosmospora vasinfecta,

Neodeightonia, Neodeightonia phoenicum,

Neoerysiphe, Neoerysiphe galeopsidis,

Neofabraea, Neofabraea perennans,

Neofusicoccum, Neofusicoccum mangiferae,

Oidiopsis, Oidiopsis gossypii,

Oidium, Oidium arachidis, Oidium caricaepapayae, Oidium indicum, Oidium mangiferae, Oidium manihotis,

Olpidium, Olpidium brassicae,

Omphalia, Omphalia tralucida,

Ophiobolus, Ophiobolus anguillides, Ophiobolus cannabinus,

Ophioirenina,

Ovulinia, Ovulinia azaleae,

Oxyporus, Oxyporus corticola,

Ozonium, Ozonium texanum,

Peltaster, Peltaster fructicola,

Penicillium, Penicillium expansum, Penicillium funiculosum,

Peniophora,

Periconia, Periconia circinata,

Periconiella, Periconiella cocoes,

Peridermium, Peridermium californicum,

Pestalosphaeria, Pestalosphaeria concentrica,

Pestalotia, Pestalotia longiseta, Pestalotia rhododendri,

Pestalotiopsis, Pestalotiopsis adusta, Pestalotiopsis arachidis, Pestalotiopsis disseminata, Pestalotiopsis guepini, Pestalotiopsis leprogena, Pestalotiopsis longiseta, Pestalotiopsis mangiferae, Pestalotiopsis palmarum, Pestalotiopsis sydowiana, Pestalotiopsis theae,

Peyronellaea, Peyronellaea curtisii,

Phacidiopycnis, Phacidiopycnis padwickii,

Phaeochoropsis, Phaeochoropsis mucosa,

Phaeocytostroma, Phaeocytostroma iliau, Phaeocytostroma sacchari,

Phaeoisariopsis, Phaeoisariopsis bataticola,

Phaeoramularia, Phaeoramularia heterospora, Phaeoramularia indica, Phaeoramularia manihotis,

Phaeoseptoria, Phaeoseptoria musae,

Phaeosphaerella, Phaeosphaerella mangiferae, Phaeosphaerella theae,

Phaeosphaeria, Phaeosphaeria avenaria, Phaeosphaeria herpotrichoides, Phaeosphaeria microscopica, Phaeosphaeria nodorum,

Phaeosphaeriopsis, Phaeosphaeriopsis obtusispora,

Phaeotrichoconis, Phaeotrichoconis crotalariae,

Phialophora, Phialophora asteris, Phialophora cinerescens, Phialophora gregata, Phialophora tracheiphila,

Phoma, Phoma clematidina, Phoma costaricensis, Phoma cucurbitacearum, Phoma destructiva, Phoma draconis, Phoma exigua, Phoma exigua, Phoma exigua var. foveata, Phoma exigua, Phoma glomerata, Phoma glycinicola, Phoma herbarum, Phoma insidiosa, Phoma medicaginis, Phoma microspora, Phoma narcissi, Phoma nebulosa, Phoma oncidiisphacelati, Phoma pinodella, Phoma sclerotioides, Phoma strasseri,

Phomopsis, Phomopsis asparagi, Phomopsis asparagicola, Phomopsis cannabina, Phomopsis coffeae, Phomopsis ganjae, Phomopsis javanica, Phomopsis longicolla, Phomopsis mangiferae, Phomopsis prunorum, Phomopsis sclerotioides, Phomopsis theae,

Phragmidium, Phragmidium mucronatum, Phragmidium rosaepimpinellifoliae, Phragmidium rubi-idaei, Phragmidium violaceum,

Phyllachora, Phyllachora banksiae, Phyllachora cannabis, Phyllachora graminis, Phyllachora gratissima, Phyllachora musicola, Phyllachora pomigena, Phyllachora sacchari,

Phyllactinia,

Phyllosticta, Phyllosticta alliariaefoliae Phyllosticta arachidishypogaeae, Phyllosticta batatas, Phyllosticta capitalensis, Phyllosticta carpogena, Phyllosticta coffeicola, Phyllosticta concentrica, Phyllosticta coryli, Phyllosticta cucurbitacearum, Phyllosticta cyclaminella, Phyllosticta erratica, Phyllosticta hawaiiensis, Phyllosticta lentisci, Phyllosticta manihotis, Phyllosticta micropuncta, Phyllosticta mortonii, Phyllosticta nicotianae, Phyllosticta palmetto, Phyllosticta penicillariae, Phyllosticta perseae, Phyllosticta pseudocapsici, Phyllosticta sojaecola, Phyllosticta theae, Phyllosticta theicola,

Phymatotrichopsis, Phymatotrichopsis omnivora,

Physalospora, Physalospora disrupta, Physalospora perseae,

Physoderma, Physoderma alfalfae, Physoderma leproides, Physoderma trifolii,

Physopella, Physopella ampelopsidis,

Pileolaria, Pileolaria terebinthi,

Piricaudiopsis, Piricaudiopsis punicae, Piricaudiopsis rhaphidophorae, Piricaudiopsis rosae,

Plenodomus, Plenodomus destruens, Plenodomus meliloti,

Pleosphaerulina, Pleosphaerulina sojicola,

Pleospora, Pleospora alfalfae, Pleospora betae, Pleospora herbarum, Pleospora lycopersici, Pleospora tarda, Pleospora theae,

Pleuroceras,

Podosphaera, Podosphaera fuliginea, Podosphaera fusca, Podosphaera leucotricha, Podosphaera macularis, Podosphaera pannosa,

Polyscytalum, Polyscytalum pustulans,

Poria, Poria hypobrunnea,

Postia, Postia tephroleuca,

Powdery mildew,

Pseudocercospora, Pseudocercospora arecacearum, Pseudocercospora cannabina, Pseudocercospora fuligena, Pseudocercosporella herpotrichoides, Pseudocercospora gunnerae, Pseudocercospora pandoreae, Pseudocercospora puderi, Pseudocercospora rhapisicoia, Pseudocercospora theae, Pseudocercospora vitis, Pseudocercosporella capsellae,

Pseudocochliobolus, Pseudocochliobolus eragrostidis,

Pseudoepicoccum, Pseudoepicoccum cocos,

Pseudopeziza, Pseudopeziza jonesii, Pseudopeziza medicaginis, Pseudopeziza trifolii,

Pseudoseptoria, Pseudoseptoria donacis,

Pucciniaceae,

Pucciniastrum, Pucciniastrum americanum, Pucciniastrum arcticum, Pucciniastrum epilobii, Pucciniastrum hydrangeae,

Pycnostysanus, Pycnostysanus azaleae,

Pyrenochaeta, Pyrenochaeta iycopersici, Pyrenochaeta terrestris,

Pyrenopeziza, Pyrenopeziza brassicae,

Ramichloridium, Ramichioridium musae,

Ramulispora, Romulispora sorghi, Romulispora sorghicoia,

Rhinocladium, Rhinocladium corticoia,

Rhizophydium, Rhizophydium graminis,

Rhizopus, Rhizopus arrhizus, Rhizopus circinans, Rhizopus microsporus, Rhizopus oryzae,

Rhytisma, Rhytisma punctatum, Rhytisma vitis,

Rigidoporus, Rigidoporus vinctus,

Rosellinia, Rosellinia arcuata, Rosellinia bunodes, Rosellinia necatrix, Rosellinia pepo,

Saccharicola, Saccharicola taiwanensis,

Schiffnerula, Schiffnerula cannabis,

Schizophyllum, Schizophyllum commune,

Schizopora, Schizopora flavipora,

Schizothyrium, Schizothyrium pomi,

Sclerophthora, Sclerophthora macrospora,

Sclerotium, Sclerotium cinnamomi, Sclerotium delphinii,

Scytinostroma, Scytinostroma galactinum,

Seimatosporium, Seimatosporium mariae, Seimatosporium rhododendri,

Selenophoma, Selenophoma linicola,

Septobasidium, Septobasidium bogoriense, Septobasidium euryaegroffii, Septobasidium gaoligongense, Septobasidium pilosum, Septobasidium polygoni, Septobasidium pseudopedicellatum, Septobasidium theae,

Septocyta, Septocyta ruborum,

Serpula, Serpula lacrymans,

Setosphaeria, Setosphaeria rostrata, Setosphaeria turcica,

Spencermartinsia, Spencermartinsia pretoriensis,

Sphaceloma, Sphaceloma arachidis, Sphaceloma menthae, Sphaceloma perseae, Sphaceloma poinsettiae, Sphaceloma sacchari, Sphaceloma theae,

Sphacelotheca, Sphacelotheca reiliana, Sphaerotheca castagnei,

Sphaerulina, Sphaerulina oryzina, Sphaerulina rehmiana, Sphaerulina rubi,

Sphenospora, Sphenospora kevorkianii,

Spilocaea, Spilocaea oleaginea,

Sporisorium, Sporisorium cruentum, Sporisorium ehrenbergii, Sporisorium scitamineum, Sporisorium sorghi,

Sporonema, Sporonema phacidioides,

Stagonospora, Stagonospora avenae, Stagonospora meliloti, Stagonospora recedens, Stagonospora sacchari, Stagonospora tainanensis,

Stagonosporopsis,

Stegocintractia, Stegocintractia junci,

Stemphylium, Stemphylium alfalfae, Stemphylium bolickii, Stemphylium cannabinum, Stemphylium globuliferum, Stemphylium lycopersici, Stemphylium sarciniforme, Stemphylium solani, Stemphylium vesicarium,

Stenella, Stenella anthuriicola,

Stigmatomycosis,

Stigmina, Stigmina carpophila, Stigmina palmivora, Stigmina plataniracemosae,

Stromatinia, Stromatinia cepivora,

Sydowiella, Sydowiella depressula,

Sydowiellaceae,

Synchytrium, Synchytrium endobioticum, Tapesia, Tapesia acuformis, Tapesia yallundae,

Taphrina, Taphrina coryli, Taphrina potentillae,

Thanatephorus, Thanatephorus cucumeris,

Thecaphora, Thecaphora solani,

Thielaviopsis, Thielaviopsis basicola, Thielaviopsis ceramica,

Thyrostroma, Thyrostroma compactum,

Tiarosporella, Tiarosporella urbis-rosarum,

Tilletia, Tilletia barclayana, Tilletia caries, Tilletia controversa, Tilletia laevis, Tilletia tritici, Tilletia walkeri,

Tilletiariaceae,

Togniniaceae,

Tranzschelia, Tranzschelia prunispinosae,

Trichoderma, Trichoderma koningii, Trichoderma paucisporum, Trichoderma songyi, Trichoderma theobromicola, Trichoderma viride,

Tubercularia, Tubercularia lateritia,

Tunstallia, Tunstallia aculeata,

Typhula, Typhula blight, Typhula idahoensis, Typhula incarnata, Typhula ishikariensis, Typhula variabilis,

Ulocladium, Ulocladium consortiale,

Uncinula,

Uredo, Uredo behnickiana, Uredo kriegeriana, Uredo musae, Uredo nigropuncta, Uredo rangelii,

Urocystis, Urocystis agropyri, Urocystis brassicae, Urocystis occulta,

Uromyces, Uromyces apiosporus, Uromyces appendiculatus, Uromyces beticoa, Uromyces ciceris-arietini, Uromyces dianthi, Uromyces euphorbiae, Uromyces graminis, Uromyces inconspicuus, Uromyces lineolatus, Uromyces musae, Uromyces oblongus, Uromyces pisi-sativi, Uromyces proëminens, Uromyces medicaginis, Uromyces trifoliirepentis, Uromyces viciae-fabae,

Urophlyctis, Urophlyctis leproides, Urophlyctis trifolii,

Ustilaginales,

Ustilago, Ustilago avenae, Ustilago esculenta, Ustilago hordei, Ustilago maydis, Ustilago nigra, Ustilago nuda, Ustilago scitaminea, Ustilago tritici,

Vankya, Vankya ornithogali,

Velvet blight,

Veronaea, Veronaea musae,

Verticillium, Verticillium alboatrum, Verticillium alfalfae, Verticillium dahliae, Verticillium isaacii, Verticillium klebahnii, Verticillium longisporum, Verticillium nonalfalfae, Verticillium theobromae, Verticillium wilt, Verticillium zaregamsianum,

Waitea, Waitea circinata,

Westea,

Wheat leaf rust,

Wheat mildew,

Wuestneiopsis, Wuestneiopsis georgiana,

Xeromphalina, Xeromphalina fraxinophila,

Zopfia, Zopfia rhizophila,

Zygosaccharomyces, Zygosaccharomyces bailiff, Zygosaccharomyces florentinus,

Zythiostroma.

Preferably, the pairs of fungi, oomycetes or bacteria vs. crop plants covered by the invention are the following:

Wheat (Triticum sativum)

Claviceps purpurea, Erysiphe graminis, Fusarium avenaceum, Fusarium culmorum, Fusarium graminearum, Fusarium langsethiae, Fusarium poae, Fusarium pseudograminearum, Gaeumannomyces graminis, Leptosphaeria nodorum, Microdochium spp., Mycosphaerella graminicola, Oculimacula acuformis, Oculimacula yallundae, Puccinia recondita, Puccinia striiformis, Pyrenophora triticirepentis, Rhizoctonia cerealis, Microdochium and Zymoseptoria tritici

Maize (Zea mays)

Fusarium graminearum, Fusarium proliferatum, Fusarium subglutinans, Fusarium verticillioides

Barley (Hordeum vulgare)

Blumeria graminis, Fusarium spp., Pyrenophora teres, Ramularia collocygni, Rhynchosporium secalis

Rice (Oryza sativa)

Cochliobolus miyabeanus, Fusarium fijikuroi, Magnaporthe oryzae, Microdochium oryzae, Pyricularia oryzae, Rhizoctonia oryzae, Rhizoctonia solani, Sarocladium oryzae, Ustilaginoides virens

Potato (Solanum tuberosum)

Alternia alternata, Alternaria solani, Phytophtora infestans, Rhizoctonia solani

Grapevine (Vinis vitifera)

Botrytis cinerea, Erysiphe necator, Plasmopara viticola, Guignardia bidwelli, Erisyphe necator, Diplodia seriata

Soya (Glycine max)

Cercopora kikuchii, Colletotrichum dematium, Corynespora cassiicola, Fusarium graminearum, Pythium spp., Rhizoctonia solani, Sclerotinia sclerotiorum, Septoria glycines

Apple tree (Malus domestica)

Monilia fructigena, Monilia laxa, Pezzicula alba, Pezzicula malicorticis, Venturia inaequalis Tomato (Lycopersicon esculentum)

Phytophtora infestans

Bean (Phaseolus vulgaris)

Uromyces appendiculatus

Radish (Raphanus sativus)

Alternaria brassicae

All fruits and vegetables

Botrytis cinerea

Strawberry plant (Fragaria sp.)

Colletotrichum acutatum

Carrot (Daucus carota)

Alternaria alternata, Alternaria cloud, Alternaria radicina

Peach (Prunus persica) and apricot (Prunus armeniaca)

Monilia fructicola, Monilia fructigena, Monilia laxa

Particularly preferably, the pairs of fungi or bacteria vs. crop plants covered by the invention are the following:

Wheat: Fusarium graminearum, Microdochium and Zymoseptoria tritici

Grapevine: Botrytis cinerea, Erysiphe necator, Plasmopara viticola, Guignardia bidwelli, Erisyphe necator, Diplodia seriata

Potato: Alternia alternata, Alternaria solani, Phytophtora infestans, Rhizoctonia solani

Tomato: Phytophtora infestans

Preparation Process

The invention also relates to a process for preparing a cell extract of one or more microalgae of the genus Amphidinium characterized by the following steps:
Harvesting fresh cells from one or more microalga(e) of the genus Amphidinium,
Optionally freezing and/or freeze-drying said cells
Suspending said fresh or frozen cells or said lyophilisate in an inorganic or organic solvent in a lyophilisate or biomass/solvent weight ratio comprised between 1:200 and 1:2.
Optionally freeze-drying the extract obtained.

“Inorganic solvent” means water and aqueous (water) solutions and “inorganic solvent” means hydrocarbon solvents (aliphatics, aromatics), oxygenated solvents (alcohols, ketones, acids, esters and ethers), halogenated solvents (dichloromethane, chloroform) and mixtures in any miscible proportions of these solvents.

Preferably, water or an oxygenated solvent, preferably C1-C4 alcohol, such as methanol or ethanol, will be used.

In the case of an organic solvent, the suspension may be carried out at a temperature between 4 and 60° C., preferably between 18 and 60° C., particularly preferably at room temperature.

Preferably, the suspension of said fresh or frozen cells or said lyophilisate in an inorganic solvent is carried out at a temperature above 60° C., preferably above 80° C., particularly preferably at a temperature above 90° C. Preferably, the suspension of said fresh or frozen cells or said lyophilisate in an inorganic solvent lasts less than 5 minutes, preferably less than 3 minutes, preferably less than 1 minute. Preferably, the temperature is then quickly returned to room temperature. Preferably, the temperature of the mixture is returned close to room temperature by placing the mixture in a cold environment, for example at a temperature close to 0° C., or by adding to the mixture an inorganic solvent at a temperature close to 0° C.

The suspension is carried out either by adding to said fresh or frozen cells or to said lyophilisate the solvent heated to the desired temperature beforehand, or the solvent is added and the resuspended mixture is adjusted to the desired temperature.

Preferably, the suspension of said fresh or frozen cells or said lyophilisate in an inorganic or organic solvent is carried out in a lyophilisate or biomass/solvent weight ratio comprised between 1:100 and 1:50.

To “quickly” return the temperature to room temperature means in less than 5 minutes, preferably in less than 3 minutes, preferably in less than 1 minute.

The invention also relates to a cell extract or a lyophilisate of one or more microalgae of the genus Amphidinium obtainable by the process for preparing a cell extract of the invention.

Advantageously, the fresh cells which are harvested and extracted come from a cell culture under conditions of temperature, photoperiod and salinity suited to the strain concerned up to a cell concentration comprised between 5·10⁴ cells/ml and 5·10⁶ cells/ml, preferentially a cell concentration comprised between 5·10⁵ cells/ml and 1·10⁶ cells/ml.

The cells are cultured for 5 to 20 days.

The light intensity is comprised between 40 μE and 200 μE, preferably comprised between 70 μE and 100 μE.

The culture temperature is generally comprised between 17° C. and 25° C.

The day/night photoperiod is preferably comprised between 8 h/16 h and 16 h/8 h.

The minimum salinity is 15 ppt.

According to a particular embodiment, Amphidinium carterae is cultivated as follows: the cells are incubated in natural or artificial seawater medium at a temperature comprised between 17 and 25° C. with a day/night cycle comprised between 8 h/16 h and 16 h/8 h, preferably 16 h/8 h.

Control Process

The invention also relates to a process for controlling crop plant and seed pathogenic fungi, oomycetes and/or bacteria, comprising applying to crop plants a cell extract of one or more microalgae of the genus Amphidinium or an extract according to the invention.

This control may be curative or preventive, preferably curative.

The invention also relates to a process for controlling crop plant and seed pathogenic fungi, oomycetes and/or bacteria, comprising the following steps:
Mixing extemporaneously in water, in a proportion of 1:4 to 1:800, a cell extract according to the invention
Applying this mixture to crop plants and/or coating said seeds with this mixture.

The invention also relates to a process for controlling crop plant and seed pathogenic fungi, oomycetes and/or bacteria, comprising the following steps:
Resuspending a lyophilizate of a cell culture of one or more microalga(e) of the genus Amphidinium at a mass concentration comprised between 5 and 500 g/L, preferentially a mass concentration comprised between 50 and 400 g/L, preferentially a mass concentration comprised between 100 and 200 g/L, in water or in an organic or inorganic solvent in a weight ratio of 1:200 to 1:2,
Extemporaneously mixing in water, in a proportion of 1:4 to 1:800
Applying the extract obtained to crop plants and/or coating said seeds with this mixture.
   In the case of suspension in water or in an inorganic solvent, the suspension is preferably carried out at a temperature above 60° C. and then the temperature is quickly returned close to room temperature by dilution in water in a proportion of 1:2 to 1:50.

Application to crop plants may be carried out by any means known to the skilled person for reaching the plant parts affected by the fungus and/or bacterium.

The extract is applied at a dose comprised between 0.005 g/L and 20 g/L, preferably between 0.5 g/L and 10 g/L, particularly preferably between 1 g/L and 5 g/L.

Seed coating may be carried out by any technique known to the skilled person allowing the active agent to remain in contact with the seed.

For example, coating may be carried out by powdering or spraying.

For example, the coating may comprise formulants and additives.

Formulants are used to allow the active substance(s) to be applied to and remain on the seed in equal and constant proportion throughout the product application process at very low doses.

Formulants include: organic solvents or water, dispersants, emulsifiers, surfactants or wetting agents, dyes, etc.

Surfactants and emulsifiers have the property of combining and stably maintaining together two incompatible liquids.

Various adjuvants may be applied to the seed. Film coatings correspond to the application of a microporous film to the seed surface. They do not change the shape or size of the seed. They improve the coverage and homogeneity of the treatment. When the farmer uses the seeds, they are easier to handle during sowing by suppressing dust and facilitating seed flow into the seeder. They improve the action of the active substance(s) under cultivation conditions. Coatings change the shape, size and weight of the seed. They improve sowing accuracy.

The treatment processes for controlling crop plan and seed pathogenic fungi and/or bacteria according to the invention are particularly suitable against Fusarium disease, preferably a Fusarium disease listed in Table 1.

TABLE 1
Summary of Fusarium diseases

| Disease name | Pathogen | EPPO code |
|---|---|---|
| Fusarium foot rot of asparagus | Fusarium culmorum | FUSACU |
| Fusarium foot rot of bean | Fusarium solani f. sp. phaseoli | FUSASH |
| Fusarium foot rot of pea | Fusarium solani f. sp. pisi | FUSASI |
| Fusarium wilt of sugarbeet | Fusarium oxysporum f. sp. betae | FUSABE |
| Fusarium wilt of potato | Fusarium coeruleum | FUSASC |
| Fusarium wilt of cabbage | Fusarium oxysporum f. sp. conglutinans | FUSACO |
| Fusarium stalk rot of maize | Gibberella fujikuroi | GIBBFU |
| Fusarium stalk rot of maize | Fusarium culmorum | FUSACU |
| Fusarium stalk rot of maize | Gibberella zeae | GIBBZE |
| Fusarium wilt of vanilla | Fusarium oxysporum f. sp. vanillae | FUSAVN |
| Fusarium wilt of pineapple | Gibberella fujikuroi var. subglutinans | GIBBFS |
| Fusarium head blight of maize | Fusarium poae | FUSAPO |
| Fusarium head blight of maize | Fusarium tricinctum | FUSATI |
| Fusarium wilt of carnation | Fusarium oxysporum f. sp. dianthi | FUSADI |
| Fusarium wilt of bromeliad | Fusarium oxysporum f. sp. aechmeae | FUSAAE |
| Fusarium wilt of gladiolus | Fusarium oxysporum f. sp. gladioli | FUSAGL |
| Fusarium head blight of cereals | Fusarium culmorum | FUSACU |
| Fusarium head blight of cereals | Gibberella rosea | FUSARO |
| Fusarium head blight of cereals | Gibberella avenacea | GIBBAV |
| Fusarium head blight of cereals | Gibberella intricans | GIBBIN |
| Fusarium head blight of cereals | Monographella nivalis | MONGNI |
| Fusarium ear rot | Gibberella zeae | GIBBZE |
| Fusarium root rot of asparagus | Fusarium oxysporum f. sp. asparagi | FUSAAS |
| Fusarium root rot of cactus | Fusarium oxysporum f. sp. opuntiarum | FUSAOP |
| Fusarium root and crown rot of tomato | Fusarium oxysporum f. sp. | FUSARL |
| | radicis-lycopersici | |
| Fusarium root and crown rot of cucumber | Fusarium oxysporum f. sp. cucumerinum | FUSACC |
| Fusarium head blight of wheat | Gibberella fujikuroi | GIBBFU |
| Fusarium wilt of cocoa | Albonectria rigidiuscula | CALORI |
| Fusarium wilt of coffee | Gibberella stilboides | GIBBST |
| Fusarium wilt of safflower | Fusarium oxysporum f. sp. carthami | FUSACA |
| Fusarium wilt of quince | Gibberella baccata | GIBBBA |
| Fusarium wilt of cucurbits | Fusarium solani f. sp. cucurbitae | FUSASU |
| Fusarium wilt of cotton | Fusarium oxysporum f. sp. vasinfectum | FUSAVA |
| Fusarium wilt of gerbera | Fusarium oxysporum f. sp. gerberae | FUSAGE |
| Fusarium wilt of gladiolus | Fusarium oxysporum f. sp. gladioli | FUSAGL |
| Fusarium wilt of flax | Fusarium oxysporum f. sp. lini | FUSALI |
| Fusarium head blight of maize | Gibberella acuminata | GIBBAC |
| Fusarium head blight of maize | Gibberella fujikuroi var. subglutinans | GIBBFS |
| Fusarium head blight of maize | Gibberella zeae | GIBBZE |
| Fusarium wilt of palm | Fusarium oxysporum f. sp. elaeidis | FUSAEL |
| Fusarium wilt of soybean | Fusarium oxysporum f. sp. glycines | FUSAGY |
| Fusarium wilt of soybean | Fusarium oxysporum f. sp. tracheiphilum | FUSATR |
| Fusarium tuber rot of potato | Gibberella cyanogena | GIBBCN |
| Fusarium root rot of rice | Gibberella fujikuroi | GIBBFU |
| Fusarium snow mould | Monographella nivalis | MONGNI |
| Fusarium dieback | Gibberella rosea | FUSARO |
| Fusarium vascular wilt | Fusarium oxysporum | FUSAOX |
| Fusarium vascular wilt of lentil | Fusarium oxysporum f. sp. lentis | FUSALE |
| Fusarium vascular wilt of watermelon | Fusarium oxysporum f. sp. niveum | FUSANV |
| Fusarium vascular wilt of tomato | Fusarium oxysporum f. sp. lycopersici | FUSALY |
| Fusarium vascular wilt of tulip | Fusarium oxysporum f. sp. tulipae | FUSATU |
| Fusarium vascular wilt of crucifers | Fusarium oxysporum f. sp. conglutinans | FUSACO |
| Fusarium vascular wilt of coffee | Gibberella xylarioides | GIBBXY |
| Fusarium vascular wilt of cabbage | Fusarium oxysporum f. sp. conglutinans | FUSACO |
| Fusarium vascular wilt of chrysanthemum | Fusarium oxysporum f. sp. chrysanthemi | FUSACH |
| Fusarium vascular wilt of cucumber | Fusarium oxysporum f. sp. cucumerinum | FUSACCC |
| Fusarium vascular wilt of cyclamen | Fusarium oxysporum var. aurantiacum | FUSAAU |
| Fusarium vascular wilt of strawberry | Fusarium oxysporum f. sp. fragariae | FUSAFR |
| Fusarium vascular wilt of bean | Fusarium oxysporum f. sp. phaseoli | FUSAPH |
| Fusarium vascular wilt of melon | Fusarium oxysporum f. sp. melonis | FUSAME |
| Fusarium vascular wilt of pea | Fusarium oxysporum f. sp. pisi | FUSAPI |
| Fusarium vascular wilt of chickpea | Gibberella baccata | GIBBBA |
| Fusarium vascular wilt of chickpea | Fusarium oxysporum f. sp. ciceris | FUSACI |
| Fusarium vascular wilt of radish | Fusarium oxysporum f. sp. raphani | FUSARA |

The treatment processes for controlling crop plant and seed pathogenic fungi, oomycetes and/or bacteria according to the invention are particularly suitable for the following pairs of fungi or bacteria vs. crop plants:
Wheat: Fusarium graminearum, Microdochium nivale and Zymoseptoria tritici
Grapevine: Botrytis cinerea, Plasmopara viticola, Guignardia bidwelli, Erisyphe necator, Diplodia seriata
Apple tree: Venturia inaegualis
Banana tree: Fusarium oxysporumand Mycosphaerella fijiensis

### EXAMPLES

### Materials & Methods

### Example 1: Microalgae Culture

The microalga Amphidinium carterae, strain AC208, comes from Algobank (Caen) and the microalgae Prymnesium parvum, strain RCC 1436, and Phaeodactylum tricornutum, strain CCMP 632, come from the Roscoff marine microorganism bank (RCC: Roscoff Culture Collection). These microalgae are cultivated in L1 artificial seawater (https://ncma.bigelow.org/algal-recipes) at 19° C. with a 12 h/12 h day/night cycle. The light intensity used is 100 μE. Biomass is recovered at the end of the exponential growth phase by centrifugation (15 min at 3000 RPM). The resulting cell pellet is frozen and then freeze-dried using a laboratory freeze-dryer (Alpha 1-2 LDplus, Labconco) for stable long-term preservation of the active substance. After freeze-drying, the dry matter is weighed.

### Example 2: Preparation of the Extract

In order to extract the active substance from the dry matter of Example 1, 20 mg of dry matter is resuspended in 1 mL of distilled water at 100° C. After incubation for 2 minutes at room temperature (20-25° C.) the extract is stored in ice and then centrifuged for 5 min at 10 000 RPM at room temperature. The supernatant containing the active substance is frozen in liquid nitrogen for long term preservation of its antifungal properties.

### Example 3: Fusarium graminearum Germination Test

Fusarium graminearum spores are grown in “mung bean” depleted medium. Spores are separated from the mycelium by filtration on Miracloth (Calbiochem), centrifuged and then resuspended at 1.6·10⁶ spores/mL. Approximately 16 000 spores are incubated in the presence of the control solution or the A. carterae extract at various concentrations. After incubation for 10 min at room temperature, the spores are placed on a slide for a germination count after 6 hours or on a petri dish for an observation of mycelium growth after 72 hours.

Tests

Extracts of various marine microorganism species belonging to three major phytoplankton phyla, dinoflagellates, haptophytes and diatoms, were tested for potential antifungal activity on cryptogamic fungi. These microalgae have the ability to produce toxins that enable them to proliferate strongly by competing with other species and are therefore potential sources of molecules that may exhibit antifungal activity. The extracts of each microalgae were obtained according to Example 2. To test the effect of these extracts on the survival of phytopathogenic fungi, the freeze-dried extracts are resuspended in water and brought into contact with a given number of Fusarium graminearum spores.

### Example 4: Inhibition of Growth and Germination of Fusarium graminearum Spores

The ability of F. graminearum to form mycelium on agar medium in the presence of these extracts is tested 72 hours later. Of the three extracts tested (Prymnesium parvum, Amphidinium carterae and Phaeodactylum tricornutum), only the Amphidinium carterae extract according to Example 2 has an inhibitory effect on mycelium formation (FIG. 1A).

To confirm this result, a dose-response test was performed by incubating the spores with the A. carterae extract according to Example 2 diluted to various concentrations (FIG. 1B). This extract proves to have fungal activity that is dose-dependent with a minimum inhibitory concentration (MIC) of 0.4 g/L (FIG. 1B).

Finally, the A. carterae extract according to Example 2 was tested to inhibit germination of F. graminearum spores. In vitro results obtained 6 hours after incubation with the extract show total inhibition of spore germination at a concentration of 2 g/L (FIG. 1C), suggesting that the extract inhibits F. graminearum spore germination and mycelium growth.

### Example 5: Effect of Freeze-Drying the A. carterae Extract on the Antifungal Activity of the Extract

The inventors determined whether or not freeze-drying the A. carterae extract inhibited antifungal activity. To that end, an A. carterae culture was extracted according to Example 2 and then half of the extract was frozen at 80° C. while the other half of the extract was freeze-dried and then resuspended in distilled water. These extracts were tested at various concentrations for their ability to inhibit F. graminearum growth according to Example 3. The results show that in both cases, frozen extract or freeze-dried extract, complete inhibition of F. graminearum growth is obtained at a concentration of 1 g/L and persists up to 5 g/L (FIG. 1D). In conclusion, freeze-drying the A. carterae extract does not affect its antifungal activity in any way.

### Example 6: Tests on Wheat Plants Infected Under Controlled Conditions

Wheat ears were inoculated with F. graminearum spores under controlled conditions and then 24 hours later the A. carterae extract of Example 2 was applied. Symptoms were read at 20 days (400° D) and 22 days (450° D) postinfection (FIG. 2A). The number of symptomatic ears (disease incidence) and the symptom score (disease severity, score 0-9) were reported in both cases: in the absence (control) and in the presence of the extract of Example 2. In comparison with the control treatment, the presence of the extract significantly reduces, by about 30%, the number of ears attacked by the disease. In addition, these ears show symptoms with a reduced severity of about 50% (FIG. 2B). These results demonstrate that the A. carterae extract of Example 2 has significant antifungal activity on the growth of wheat phytopathogenic fungi under in vitro and in planta conditions.

### Example 7: Tests on Several Families of Grapevine Phytopathogens: Botrytis cinerea, an Ascomycete Responsible for Grey Rot, Plasmopara viticola, an Oomycete Responsible for Downy Mildew, Erisyphe necator, an Ascomycete Responsible for Powdery Mildew and Diplodia seriata, One of the Causal Agents of the Wood Disease Esca

At a concentration of 1 g/L, the A. carterae extract of Example 2 totally inhibits P. viticola growth on detached leaves (FIG. 3A, left side), while under the same conditions the extract has no effect on E. necator growth (FIG. 3A, right side). In vitro tests were conducted on Botrytis cinerea indicating that the extract totally inhibits B. cinerea growth at a concentration of 5 g/L (FIG. 3B). The antifungal activity of the extract on B. cinerea was confirmed on detached grapevine leaves (FIG. 3B). In addition, in vitro tests were carried out on the different families of fungi responsible for grapevine esca. The results show that the extract at a concentration of 2 g/L strongly inhibits the growth of these fungi (FIG. 3C).

### Example 8: In Vitro Tests on Microdochium majus, Fusarium Graminearum, Zymoseptoria Tritici, Fusarium Oxysporum, Rhizoctonia Solani and Phytophthora infestans

In order to determine whether the A. carterae extract according to Example 2 has antifungal activity on a broad spectrum of plant fungi, tests were performed on other fungi responsible for Fusarium disease: Fusarium oxysporum (Fusarium wilt of the banana tree) and Microdochium (Fusarium head blight of wheat) as well as on another important wheat disease: Septoria blotch disease caused by Zymoseptoria tritici. Tests were also carried out on two potato pathogens: the basidiomycete Rhizoctonia solani and the oomycete Phytophthora infestans. In all cases, the A. carterae extract according to Example 2 strongly limited the growth of fungi and oomycetes under in vitro conditions (Tables 2 to 6).

TABLE 2
Efficacy of the microalgae extract in vitro against macroconidia of F. graminearum cultivated
on PDB medium at 25° C. and in the dark during 24 and 72 hours of incubation.

| Incubation | Fungicidal efficacy (% of untreated control)^{a} | | | | | | | | |
|---|---|---|---|---|---|---|---|---|---|
| time | 0.010 mg/ml | 0.025 mg/ml | 0.050 mg/ml | 0.125 mg/ml | 0.250 mg/ml | 0.375 mg/ml | 0.5 mg/ml | 1.25 mg/ml | 5.0 mg/ml |
| 24 h | 2.0 | 5.9 | 14.3 | 56.2 | 100.0 | 100.0 | 100.0 | 100.0 | 100.0 |
| 72 h | 0.3 | 0.3 | 0.2 | 1.1 | 100.0 | 100.0 | 100.0 | 100.0 | 100.0 |

^{a}The fungicidal efficacy of the microalgae extract was determined after 24 and 72 hours of incubation on PDB medium at 25° C. and in the dark by measuring the optical density at 590 nm in each well of the 96-well microplate. Each value corresponds to the average of 3 repetitions per condition tested

TABLE 3
Efficacy of the microalgae extract in vitro against Zymoseptoria tritici strain Mg StA,
Rhizoctonia solani strain Rsol AG3, Microdochium majus strain Mmaj E11 and Fusarium graminearum
strain Fg 1.

| | Fungicidal efficacy (% of | | | | | | |
|---|---|---|---|---|---|---|---|
| | untreated control)^{a} | | | | | | |
| Pathogenic agent | 0.050 mg/ml | 0.125 mg/ml | 0.250 mg/ml | 0.5 mg/ml | 1.25 mg/ml | 2.5 mg/ml | 5.0 mg/ml |
| Z. tritici | 5.0 | 98.4 | 100.0 | 100.0 | 100.0 | 100.0 | 100.0 |
| R. solani | 5.7 | 71.1 | 100.0 | 100.0 | 100.0 | 100.0 | 100.0 |
| M. majus | 16.2 | 42.4 | 100.0 | 100.0 | 100.0 | 100.0 | 100.0 |
| F. graminearum | 15.8 | 31.7 | 98.9 | 100.0 | 100.0 | 100.0 | 100.0 |

^{a}The fungicidal efficacy of the microalgae extract was determined after 72 hours of incubation for M. majus and F. graminearum or 5 days of incubation for Z. tritici and R. solani on PDB medium at 25° C. and in the dark by measuring the optical density at 590 nm in each well of the 96-well microplate. Each value corresponds to the average of 3 repetitions per condition tested.

TABLE 4
ED₅₀ and MIC (mg active substance/ml) of the microalgae extract
against Zymoseptoria tritici strain Mg StA, Rhizoctonia solani
strain Rsol AG3, Microdochium majus strain Mmaj E11 and
Fusarium graminearum strain Fg 1.

| | Fungicidal efficacy | |
|---|---|---|
| | (mg active substance/ml)^{a} | |
| Pathogenic agent | ED₅₀ | MIC |
| Z. tritici | 0.084 ± 0.010 | 0.126 ± 0.006 |
| R. solani | 0.106 ± 0.015 | 0.189 ± 0.041 |
| M. majus | 0.123 ± 0.009 | 0.266 ± 0.019 |
| F. graminearum | 0.136 ± 0.006 | 0.290 ± 0.003 |

^{a}ED₅₀: Effective dose of the microalgae extract reducing the development of the tested pathogens by 50%; MIC (minimum inhibitory concentration): the lowest concentration that inhibits the development of the tested pathogens by 100%. Each value corresponds to the average of 3 repetitions per condition tested ± standard deviation.

TABLE 5
Efficacy of the microalgae extract in vitro against Phytophthora infestans strain PiF14,
Fusarium oxysporum f. sp. cubense strain CBS 102013.

| | Fungicidal efficacy (% of untreated control)^{a} | | | | | | |
|---|---|---|---|---|---|---|---|
| Pathogenic agent | 0.050 mg/ml | 0.125 mg/ml | 0.250 mg/ml | 0.5 mg/ml | 1.25 mg/ml | 2.5 mg/ml | 5.0 mg/ml |
| P. infestans | 2.1 | 4.4 | 8.7 | 35.9 | 75.8 | 98.7 | 100.0 |
| F. oxysporum f. sp. cubense | 0.0 | 14.0 | 100.0 | 100.0 | 100.0 | 100.0 | 100.0 |

^{a}The fungicidal efficacy of the microalgae extract was determined after 72 hours of incubation for the 3 pathogens at 20° C. and in the dark by measuring the optical density at 590 nm in each well of the 96-well microplate. Each value corresponds to the average of 3 repetitions per condition tested.

TABLE 6
ED₅₀ and MIC (mg active substance/ml) of the microalgae
extract against Phytophthora infestans strain PiF14, Fusarium oxysporum
f. sp. cubense strain CBS 102013

| | Fungicidal efficacy (mg active substance/ml)^{a} | |
|---|---|---|
| Pathogenic agent | ED50 | MIC |
| P. infestans | 0.70 ± 0.040 | 5.0 ± 0.0 |
| F. oxysporum f. sp. | 0.175 ± 0.02 | 0.25 ± 0.03 |
| cubense | | |

^{a}ED₅₀: Effective dose of the microalgae extract reducing the development of the tested pathogens by 50%; MIC (minimum inhibitory concentration): the lowest concentration that inhibits the development of the tested pathogens by 100%. Each value corresponds to the average of 3 repetitions per condition tested ± standard deviation.
^{B}n.i.: No inhibition, even at the highest tested concentration of 5 mg/ml.

### Example 9: In Vitro and in Planta Test (Apple Plantlets) Against Apple Scab (Venturia inaequalis)

The antifungal activity of extract D at 5 g/L was tested on a petri dish in the presence of Venturia inaequalis spores deposited 2 hours after or 3 hours before extract D. After 48 hours of incubation, the percentage of spore germination was evaluated. The presence of extract D, 2 hours before or 3 hours after inoculation, completely inhibits V. inaequalis spore germination unlike the presence of water.

The in planta tests on apple plantlets were carried out according to the following protocol:

2 modes tested: spraying of extract D at 5 g/L 2 hours before inoculation (ExtractBefore) and spraying of extract D at 5 g/L 3 hours after inoculation (ExtractAfter)

3 control modes:
2 untreated control modes (spraying of water on the same dates as the product) (WaterBefore and WaterAfter)
1 mode with a reference fungicide (captan)

Artificial inoculation of plants with a Venturia inaequalis strain at 100 000 spores/ml.

Scab symptoms noted at 4 dates post-inoculation (visual estimate of % scab area)

Biological material:
Plantlets derived from roughly 3-week-old apple seedlings in soil balls—from pips taken from the Gala variety (apples harvested in an orchard planted with Gala and Elstar)
Venturia inaequalis spores on cellophane—Strain EUB04

3 boxes of 12 plantlets for each mode

D+9, D+14, D+17, D+21: Notation of percentage of leaf area affected (from 0 to 100% in 10% increments)

Since the normality and homogeneity conditions of the variances of the residues were not met, a non-parametric Kruskal-Wallis test was performed. This test being significant, the medians of the different modes were compared pairwise using the Nemenyi test based on Tukey's distance. These analyses were carried out with the R software (version 3.1.2).

Application of the extract of Example 2 did not result in phytotoxicity to the plants: no necrosis, chlorosis or blister reactions were observed.

Observation at D9: no scab symptoms on the 5 modes.

On the untreated controls (WaterBefore and WaterAfter), scab developed to reach a median of 50 to 60% at D21. No significant difference between the two water modes regardless of the date.

On the control treated with captan, no scab development was observed.

On the treated modes (ExtractBefore and ExtractAfter), scab development is significantly lower than for the untreated controls regardless of the observation date. Regardless of the observation date, there is no significant difference between the ExtractBefore mode and the captan mode. 14 days after inoculation, there is also no significant difference between the ExtractAfter mode and the captan mode, but at 17 and 21 days post-inoculation there is significantly more scab for the ExtractAfter mode than for the captan mode. There are no significant differences between ExtractBefore and ExtractAfter.

Very good efficacy of the A. carterae extract against scab when applied 2 hours before inoculation or 3 hours after inoculation.

### Example 10: In Planta Test on Different Colletotrichum Species

To determine whether the A. carterae extract has a biocidal effect on the ascomycete Colletotrichum, two different Colletotrichum species were tested: Colletotrichum fructicola and Colletotrichum orbiculare. For these two species, the procedure is identical: a volume of extract at a given concentration (from 1 to 10 g/L), or a volume of water, was mixed with a volume containing 2·10⁶ Colletotrichum fructicola spores or 2·10⁶ Colletotrichum orbiculare spores, then 10-μl spots were applied on detached strawberry or cucumber leaves, respectively. The leaves were kept in a petri dish in a humid atmosphere for 6 days at 24° C. in a culture chamber (day/night; 14 h/12 h). The results show that the A. carterae extract totally inhibits the growth of these two Colletotrichum species from a concentration of 1 g/L.

### Example 11: Wall and Plasma Membrane Integrity Test of Fusarium graminearum Conidiospores

F. graminearum conidiospores are incubated for 1 hour in the presence of the extract according to Example 2 (5 g/L) or the same extract (5 g/L) inactivated by a temperature above 60° C. Propidium iodide is added to determine the membrane integrity of the spores.

The photographs show propidium iodide staining of the conidiospores in the presence of the extract according to Example 2 (5 g/L) and not in the presence of the same inactivated extract (5 g/L).

These results show that the extract according to Example 2 acts by significantly increasing the porosity of the wall and the plasma membrane of Fusarium graminearum conidiospores.

All these results show that the extract derived from A. carterae has a substantial antifungal activity, whether under in vitro or in planta conditions, on a broad spectrum of phytopathogenic fungi.

### Example 12: HPLC Fractionation of Extract D and Test of Antifungal Activity of the Different Fractions Obtained

In an attempt to characterize which molecules present in the A. carterae extract are responsible for antifungal activity, the bioguided fractionation strategy was selected: the A. carterae extract is fractionated on an HPLC column and tests of biocidal activity against F. graminearum spores are performed for each fraction to determine which fraction contains the molecule(s) responsible for the antifungal activity.

200 mg of freeze-dried cells are solubilized in 1 mL of methanol. After centrifugation for 10 min at 4400 rpm, the supernatant is recovered and the total alcohol liquid phase recovered is filtered on paper, the solution is then evaporated in a rotary evaporator at low temperature to collect 48.5 mg of extracted product. An activity test is performed on F. graminearum spores to confirm the activity of the A. carterae extract (FIG. 4A).

The extract being active, semi-preparative fractionation experiments were performed on a reversed-phase C18 column using a Thermo Scientific Ultimate 3000 high-performance liquid chromatograph according to the following protocol:
The A. carterae extract was dissolved at 5 g/L in methanol. The following conditions were applied for the extract:
Flow rate: 2.5 mL/min.
Column: reversed-phase C₁₈: L=250 mm; I.D.=10 mm; P.D.=5 μm.
Injection volume: 150 μL.
Injection temperature: 24° C.
Detection wavelength: 280 nm.
Gradient programme described in Table 7 below.

TABLE 7
Optimal semi-preparative solvent gradient programme.

| Time (min) | Solvent A (%) | Solvent B (%) |
|---|---|---|
| 0 | 50 | 50 |
| 25 | 0 | 100 |
| 40 | 0 | 100 |
| 43 | 50 | 50 |
| 48 | 50 | 50 |

Solvent A: Milli-Q water + 0.1% formic acid; Solvent B: Methanol.

The chromatogram obtained under these conditions is presented in FIG. 4B. Five fractions, F1 to F5, were formed, as described in FIG. 4B, so that each majority peak corresponds to a fraction. Activity tests on F. graminearum growth were performed with 5 mg/ml of each fraction. The results indicate that only fraction F1 still has biocidal activity (FIG. 4C); an MIC of 0.75 mg/ml was determined for this fraction (FIG. 4D).

Fraction F1 was subjected to a new fractionation according to the following protocol:

Extract F1 was dissolved at 5 g/L in methanol. The following conditions were applied for extract D:
Flow rate: 2.5 mL/min.
Column: reversed-phase C₁₈: L=250 mm; I.D.=10 mm; P.D.=5 μm.
Injection volume: 150 μL.
Injection temperature: 24° C.
Detection wavelength: 280 nm.
Gradient programme described in Table 8 below

TABLE 8
Optimal semi-preparative solvent gradient programme.

| Time (min) | Solvent A (%) | Solvent B (%) |
|---|---|---|
| 0 | 30 | 70 |
| 10 | 15 | 85 |
| 15 | 15 | 85 |
| 25 | 0 | 100 |
| 30 | 0 | 100 |
| 32 | 30 | 70 |
| 35 | 30 | 70 |

Solvent A: Milli-Q water + 0.1% formic acid; Solvent B: Methanol.

The chromatogram obtained under these conditions is presented in FIG. 5A. Five fractions, F1-1 to F1-5, were formed, as described in FIG. 5A, the majority peak corresponding to fraction F1-2. Activity tests on F. graminearum growth were performed with 5 mg/ml of each fraction. The results indicate that only fractions F1-2 and F1-3 have biocidal activity (FIG. 5B); an MIC of 0.75 mg/ml was determined for fraction F1-2 (FIG. 5C).

### Example 13: Mass Spectrometric Analysis of Fraction F1-2

To better characterize the molecule present in fraction F1-2, a mass spectrometric analysis was performed under the following conditions:

The experiments were performed in infusion mode on a QStar Elite spectrometer (Applied Biosystems).

Ionization mode: Positive-mode electrospray (ESI)
Electrospray needle voltage: 4500 V at room temperature
Injection conditions: 20 μL of sample dissolved in methanol, with a methanol flow rate of 400 μL/min
Scan range: 100 to 2000 daltons
FIG. 6A shows the mass spectrum acquired by positive-mode electrospray ionization on the molecule of interest collected F1-2.
The exact mass determined is 1381.8276 daltons. It corresponds to a sodium adduct formed during ionization ([M+Na]⁺). After subtracting the mass of sodium, 23 daltons, the mass of the molecular peak of the compound is 1358.8 Da.
A thorough analysis of the exact mass makes it possible to determine one or more empirical formulas related to this mass, with an error of 5 ppm. After rejecting unrealistic proposals, the empirical formula selected is C₇₁H₁₂₂O₂₄.
A thorough analysis by tandem mass spectrometry was performed on this peak at 1381.8276 Da. The mass spectrum is presented in FIG. 6B. Several peaks (circled peaks FIG. 6B) are similar to the peaks resulting from the fragmentation of amphidinol 18 (7).

### Example 14: Nuclear Magnetic Resonance (NMR) Analysis of Fraction F1-2

To determine the structure of the molecule with a molecular weight of 1358.8 Da, an NMR analysis was performed according to the following procedure:

Samples corresponding to peak F1-2 were collected and then completely dissolved in about 350 μL of deuterated methanol (MeOD₄).

The experiments were performed on a Bruker Avance 14.1T spectrometer equipped with a multinuclear probe. One- and two-dimensional proton and carbon spectra were acquired using pulse sequences available from the Bruker sequence library.

The acquisition conditions are as follows:
¹H: Number of scans: 512; Pulse: 8 μs; Acquisition: 5.0 s; Relaxation: 1.0 s
¹³C DEPT135: Number of scans: 44666; Pulse: 12 μs; Acquisition: 1.0 s; Relaxation: 3.0 s
HSQC: Number of scans: 64; 512 increments
HMBC: Number of scans: 48; 512 increments
COSY: Number of scans: 56; 256 increments
TOCSY: Number of scans: 48; 256 increments
The ¹³C DEPT135 sequence is an experiment for sorting carbons according to the number of directly bound protons: CH₃ and CH>0 and CH₂<0
The correlation spectroscopy (COSY) sequence is a 2D homonuclear experiment for identifying protons in scalar interaction spaced 2 or 3 bonds apart.
The total correlation spectroscopy (TOCSY) sequence is a 2D homonuclear experiment for identifying protons in scalar interaction spaced 3 or more bonds apart.
The heteronuclear single quantum correlation (HSQC) sequence is a 2D heteronuclear experiment that shows the direct interactions between a carbon and the directly linked proton(s).
The heteronuclear multiple bond correlation (HMBC) sequence is a 2D heteronuclear experiment that shows the correlations between protons and carbons separated by intervals of 2 or 3 bonds.
The 1D spectra were processed by a Fourier transform. The spectra were processed by a Fourier transform in both dimensions.
Based on the results obtained, the acquired ¹H spectrum, shown in FIG. 7A, shows a series of peaks distributed over a large spectral window comprised between 1 and 6 ppm. This confirms that the compound studied contains aliphatic and olefinic protons. These shifts also suggest the presence of heteroatoms such as oxygen.
The ¹³C DEPT135 spectrum (FIG. 7B) shows a series of peaks in the spectral window comprised between 13 and 211 ppm. The HMBC and HSQC 2D sequences show how the carbons are chained together. The COSY and TOCSY 2D sequences confirm the structural sequence based on proton shifts and correlations.
The number of unsaturations and rings observed is consistent with that calculated for the molecule on the basis of its empirical formula (=11):

Nᵢ=(2n_{C}+2−n_{H}+n_{N}−n_{X})/2
With: n_{C}: the number of carbon atoms, n_{H}: the number of hydrogen atoms, n_{N}: the number of nitrogen atoms, n_{X}: the number of halogen atoms.
The 11 unsaturations are distributed as follows:
   1 ketone function
   8 double bonds, including 2 chain-end
   2 rings
The COSY and TOCSY sequences made it possible to reconstruct the entire carbon skeleton of the structure. These correlations are indicated by links in bold in FIG. 8. These results show that the secondary structure of the molecule corresponds to that of amphidinol 18. The overall interpretation was confirmed by comparing the results obtained in this study with those found in the literature (7).
In conclusion, all results obtained by nuclear magnetic resonance and mass spectrometry confirm that the molecule present in fraction F1-2, obtained after fractionation of extract D, and which has antifungal activity against F. graminearum, is the amphidinol 18 molecule whose specific chemical data are as follows:
   Empirical formula: C₇₁H₁₂₂O₂₄
   Molar mass: 1358.83 g·mol⁻¹

### REFERENCES

(1) Arseniuk, E., Foremska, E., Goral, T., Chelkowski, J. 1999. Fusarium head blight reactions and accumulation of deoxynivalenol (DON) and some of its derivatives in kernels of wheat, triticale and rye. Journal of Phytopathology 147, 577-590
(2) Devi P, Wahidulla S, Kamat T and D'Souza L (2011). Screening marine organisms for antimicrobial activity against clinical pathogens. Indian J. Geomar. Sci. (40) 338-346.
(3) Mayer A M, Rodriguez A D, Taglialatela-Scafati O, Fusetani N (2013). Marine pharmacology in 2009-2011: marine compounds with antibacterial, antidiabetic, antifungal, antiinflammatory, antiprotozoal, antituberculosis, and antiviral activities; affecting the immune and nervous systems, and other miscellaneous mechanisms of action. Mar Drugs. 11(7):2510-73
(4) Bowler, C., Vardi, A., & Allen, A. E. (2010). Oceanographic and biogeochemical insights from diatom genomes. Annual Review of Marine Science, 2, 333-65. doi:10.1146/annurev-marine-120308-081051
(5) Murray S, Garby T, Hoppenrath M, Neilan BA (2012). Genetic diversity, morphological uniformity and polyketide production in dinoflagellates (Amphidinium, Dinoflagellata). PLoS One. 7(6)
(6) Morsy N, Houdai T, Matsuoka S, Matsumori N, Adachi S, et al. (2006). Structures of new amphidinols with truncated polyhydroxyl chain and their membrane-permeabilizing activities. Bioorganic and Medicinal Chemistry 14: 6548-6554.
(7) Nuzzo G, Cutignano A, Sardo A, Fontana A (2014). Antifungal Amphidinol 18 and its 7-sulfate derivative from marine dinoflagellate Amphidinium carterae, J. Nat. Prod. 1524-1527.

1. A process for controlling crop plant and seed pathogenic fungi, oomycetes, and/or bacteria comprising applying to crop plants and/or coating said seeds with a cell extract of one or more microalgae of the genus Amphidinium for its fungicidal and/or bactericidal activity.

2. A process according to claim 1, wherein the one or more microalgae of the genus Amphidinium is Amphidinium carterae.

3. A process according to claim 1, said extract comprising amphidinol 18 or amphidinol 19.

4. A process according to claim 1, wherein said crop plant and seed pathogenic fungi are fungi pathogenic to crop plants and seeds of the genera Fusarium, Colletotrichum, Mycosphaerella, Phytophthora, and Alternaria.

5. A process according to claim 4, wherein said fungi pathogenic to crop plants and seeds of the genera Fusarium, Colletotrichum, Mycosphaerella, Phytophthora, and Alternaria are selected from the group consisting of Fusarium oxysporum, Fusarium solani, Fusarium avenaceum, Fusarium culmorum, Fusarium graminearum, Fusarium moniliforme, Fusarium poae, Fusarium proliferatum, Fusarium sporotrichioides, Fusarium subglutinans, Fusarium tricinctum, Colletotrichum acutatum, Colletotrichum graminicola, Colletotrichum coffeanum, Colletotrichum gloeosporioides, Mycosphaerella graminicola, Phytophthora infestans, Alternaria solani, and Alternaria brassisicola.

6. A process for preparing a cell extract of one or more microalga(e) of the genus Amphidinium characterized by the following steps:
harvesting fresh cells from one or more microalga(e) of the genus Amphidinium; and
resuspending said lyophilisate or said fresh or frozen cells in an inorganic or organic solvent in a weight ratio of 1:200 to 1:50.

7. The process according to claim 6, wherein the fresh cells are harvested at a cell concentration comprised between 5·10⁴ cells/ml and 5·10⁶ cells/ml.

8. A cell extract or lyophilisate of Amphidinium cells obtainable by the process of claim 6.

9. A process for controlling crop plant and seed pathogenic fungi, oomycetes, and/or bacteria comprising applying to crop plants and/or coating said seeds with a cell extract or lyophilisate according to claim 8.

10. A process for controlling crop plant and seed pathogenic fungi, oomycetes, and/or bacteria, comprising the following steps:
mixing extemporaneously in water, in a proportion of 1:4 to 1:800, a cell extract of one or more microalga(e) of the genus Amphidinium or an extract according to claim 8; and
applying this mixture to crop plants and/or coating said seeds with this mixture.

11. A process according to claim 1, comprising the following steps:
resuspending a lyophilisate of a cell culture of one or more microalga(e) of the genus Amphidinium at a mass concentration comprised between 5 and 500 g/L in water or in an organic or inorganic solvent in a weight ratio of 1:200 to 1:2,
extemporaneously mixing in water, in a proportion of 1:4 to 1:800, and
applying the extract obtained to crop plants and/or coating said seeds with this mixture.

12. A process according to claim 2, said extract comprising amphidinol 18 or amphidinol 19.

13. A cell extract or lyophilisate of Amphidinium cells obtainable by the process of claim 7.

14. The process according to claim 4, wherein said crop plant and seed pathogenic fungi are the pairs of fungi vs. crop plants: Triticum sativum/Mycosphaerella graminicola, Triticum sativum/Fusarium graminearum, Solanum tuberosum/Phytophtora infestans, Vinis vitifera/Plasmospora viticola, Vinis vitifera/Erysiphe necator, and/or Lycopersicon esculentum/Phytophtora infestans.

15. A process according to claim 11, wherein said lyophilisate of a cell culture of one or more microalga(e) of the genus Amphidinium is resuspended at a mass concentration comprised between 50 and 400 g/L.

16. A process according to claim 11, wherein said lyophilisate of a cell culture of one or more microalga(e) of the genus Amphidinium is resuspended at a mass concentration comprised between 100 and 200 g/L.

17. A process according to claim 6, further comprising: (1) freezing and/or freeze-drying said cells, and/or (2) freeze-drying said extract.

18. A process according to claim 17, wherein the fresh cells are harvested at a cell concentration comprised between 5·10⁴ cells/ml and 5·10⁶ cells/ml.

19. A cell extract or lyophilisate of Amphidinium cells obtainable by the process of claim 17.
